(19) United States
(12) Patent Application Publication
(10) Pub. No.: US 2015/0079061 A1
(43) Pub. Date: Mar. 19, 2015

(54) METHOD FOR THE HARVESTING, PROCESSING, AND STORAGE OF PROTEINS FROM THE MAMMALIAN FETO-PLACENTAL UNIT AND USE OF SUCH PROTEINS IN COMPOSITIONS AND MEDICAL TREATMENT

(71) Applicants: Patrick J. Casey, Kumeu (NZ); Murray Hoare, Kumeu (NZ)

(72) Inventors: Patrick J. Casey, Kumeu (NZ); Murray Hoare, Kumeu (NZ)

(21) Appl. No.: 14/330,818

(22) Filed: Jul. 14, 2014

Related U.S. Application Data
(60) Provisional application No. 61/845,682, filed on Jul. 12, 2013.

(51) Int. Cl. A61K 35/54; A61K 38/17; A61K 35/50

(52) U.S. Cl. CPC A61K 35/54; A61K 35/50; A61K 38/1709
USPC 424/94.2; 424/551; 424/553; 424/559; 424/570; 424/571; 424/582; 424/583

(57) ABSTRACT

The invention provides a method of harvesting, processing and storing a plurality of proteins from a mammalian feto-placental unit. The method includes dissecting a mammalian uterus to harvest at least one component of the mammalian feto-placental unit; blast freezing the component; and storing the blast frozen component; wherein the blast frozen component includes the proteins. In one embodiment, the method includes lyophilizing the blast frozen component to remove at least some water from the blast frozen component thereby creating a freeze-dried form; and storing the lyophilized component; wherein the lyophilized component includes the proteins. The invention also provides a composition including proteins from at least one lyophilized, blast frozen component of a harvested mammalian feto-placental unit reconstituted with a fluid. The invention further provides method of treatment of a disease or aging in a mammalian subject including administering to the mammalian subject a plurality of proteins from at least one blast frozen component harvested from a mammalian feto-placental unit; and reducing an accumulation of at least one intracellular protein in the subject.

### STATEMENT REGARDING FEDERALLY SPONSORED RESEARCH OR DEVELOPMENT

Not Applicable

### BACKGROUND OF THE INVENTION

The present invention broadly relates to methods of harvesting mammalian feto-placental proteins and in particular, chaperone proteins, for use in compositions and medical therapies for the treatment of disease or aging in mammals, and in particular, to humans.

Aging, which is both chronological and determined by cellular processes, is associated with an increase in disease and pathological processes. The causes of aging are unknown, but recent reviews correlate aging in humans with the instability of mitochondria leading to an increase in intra-cellular reactive oxygen species (ROS), potential shortening of the telomere, protein dysfunction and/or cellular death. The increase in ROS in turn appears to adversely affect DNA, mRNA, and protein synthesis, folding, transport and degradation. The culmination of these events in turn leads to apoptosis, i.e., cellular “suicide”, followed by necrosis, i.e., cellular death.

Some research has focused on mechanisms which initiate or allow the up-regulation of intra-cellular ROS to determine whether the mechanisms are sudden or cumulative and whether the mechanism can be slowed to any extent. Research has also investigated the role of chaperone proteins in responding to increased ROS. Notably, aging appears to decrease the intra-cellular and extra-cellular levels of chaperone proteins. For the purposes of this application, chaperone proteins, which include pharmacological chaperones, pharmacoperones, and pharma-cochaperones, are defined as target-specific, small molecules that bind to their target proteins to facilitate biogenesis and/or prevent and/or correct protein misfolding. Pharmacological chaperoning: a primer on mechanism and pharmacology, Leidenheimer Nancy J., Ryder Katelyn G., Pharmacol Res. 2014 May; 83:10-9. doi: 10.1016/j.phrs.2014.01.005. Epub 2014 Feb. 14. Chaperone protein targets include enzymes, receptors, transporters, and ion channels. Id. Chaperone proteins prevent the accumulation of misfolded proteins by promoting their refolding, degeneration and/or exocytosis. Keep your heart in shape: molecular chaperone networks for treating heart disease, Tarone Guido, Brancaccio Mara, Cardiovasc Res. 2014 Jun. 1; 102(3):346-61. doi: 10.1093/cvd/cvu049. Epub 2014 Feb. 28., PMID:24585203 [PubMed—in process]. Chaperone proteins also play a role in intracellular signaling by controlling conformational changes required for the activation and deactivation of signaling proteins and assembly in signalosome complexes. Id.

Today, chaperone proteins are not readily available for medical research and possible medical therapies, however. Thus, there is a need for methods of harvesting, processing and storing chaperone proteins. In addition, there is a need to develop compositions and medical therapies which make use of chaperone proteins in the treatment of disease or aging, and in particular, disease related to protein dysfunction.

### BRIEF SUMMARY OF THE INVENTION

In one aspect, the invention provides a method of harvesting, processing and storing a plurality of proteins from a mammalian feto-placental unit. The method includes dissecting a mammalian uterus to harvest at least one component of the mammalian feto-placental unit; blast freezing the component; and storing the blast frozen component; wherein the blast frozen component includes the proteins. In one embodiment, the method includes lyophilizing the blast frozen component to remove at least some water from the blast frozen component thereby creating a freeze-dried form; and storing the lyophilized component; wherein the lyophilized component includes the proteins. In an additional embodiment, the proteins include at least one essential fragment of at least one of the proteins. In a further additional embodiment, the proteins include a plurality of chaperone proteins.

In another embodiment, the proteins are selected from the group consisting of Serum albumin, Actin, cytoplasmic 1, I alpha globin, Hemoglobin fetal subunit beta, Vimentin, Beta globin chain, TPM1, Annexin A2, Protein disulfide isomerase family A member 3, Alpha-2-HS-glycoprotein, Fatty acid binding protein 5, Cofilin-1, 78 kDa glucose-regulated protein, Gelsolin isoform b, Beta-A globin chain, Glyceraldehyde-3-phosphate dehydrogenase, Heat shock protein alpha, Heat shock protein 70, Peptidylprolyl isomerase A, 14-3-3 protein zeta/delta, Histone H3, Peroxiredoxin 2, Cathepsin D, Uterine milk protein, Tubulin beta chain, Myosin light chain 6, Endoplasmic reticulum protein 29, Tubulin alpha chain, Solute carrier family 2, facilitated glucose transporter member 1, Alpha-1-antitrypsin transcript variant 1, Heat shock protein 10, Pregnancy-associated glycoprotein 3, Hemoglobin subunit beta, Isocitrate dehydrogenase [NADP] cytoplasmic, Elongation factor-1 alpha, Phosphoglycerate kinase, 14-3-3 protein epsilon, Putative tropomyosin, Tumor protein translationally-controlled 1, Galectin-1, Transaldolase 1, Pregnancy-associated glycoprotein 4, Pregnancy-associated glycoprotein 1, Sodium/potassium-transporting ATPase subunit alpha-1, Lamin B1, pregnancy-associated glycoprotein 6, 14-3-3 protein beta/alpha, metallopeptidase inhibitor 2, fatty acid binding protein 5, Myosin regulatory light chain MRCL3, Transferrin, Enolase 1, Cathelicidin-1, 6-phosphogluconate dehydrogenase decarboxylating, Elongation factor-1 alpha, ATP-citrate synthase, Ribosomal protein S8, Pyruvate kinase, Pre-mRNA splicing factor SRP20-like protein, alpha 2, 5 prime, Malate dehydrogenase, Cystatin-B, Chorionic somatomammotropin hormone, Carbonic anhydrase 2, SLC25A6, Decorin, 60S ribosomal protein L6, Protein disulfide isomerase-associated 4, Pregnancy-associated glycoprotein 11, Prostaglandin F synthase, Integrin beta-1, H+ transporting ATP synthase subunit D, RHOA, Adenylate kinase, Lactate dehydrogenase A, RAB10, Glucose-6-phosphate 1-dehydrogenase, Elongation factor 1-delta, Ribosomal protein S17, Insulin-like growth factor-binding protein-7, Ribosomal protein L19, ATP synthase alpha subunit, RAC1, Calpain II 80 kDa subunit, Secreted phosphoprotein 24, CD9 antigen, Aspartate aminotransferase, DNA mismatch repair protein MutL, Ribosomal protein S6, 14-3-3 protein gamma LDHA protein, Putative peptidase, Myosin light chain kinase, smooth muscle, cAMP-dependent protein kinase regulatory subunit alpha 1, Elongation factor 1-alpha, Actin, GNAZ, Eukaryotic translation initiation factor 5A, Mitochondrial bifunctional methylenetetrahydrofolate dehydrogenase/cyclohydrolase, Solute carrier family 2, facilitated glucose transporter member 3, Thioredoxin, ADP-ribosylation factor 1, NADH dehydrogenase (Ubiquinone) 1 beta, Proteasome subunit alpha type, 2, Gamma fibrinogen, Putative H-ATPase subunit B, Proteasome subunit alpha type, 60S ribosomal protein L10, 14-3-3 protein sigma, Chaperone protein DnaK, Ribosomal protein s15, Putative uncharacterized protein, Aspartyl-tRNA synthetase, and Proteasome subunit alpha.

In another embodiment, the method includes using at least a portion of the proteins for medicinal purposes. In yet another embodiment, the step of using at least a portion of the proteins for medicinal purposes includes treating for a disease or aging in a mammalian subject. In still other embodiments, the treating step includes administering the portion of the proteins to the mammalian subject with at least one of a sublingual procedure, an intra-ocular procedure, an intra-rectal procedure, and an intra-gastro-intestinal procedure.

In an additional embodiment, the treating step includes reconstituting the portion of the proteins of the lyophilized component with a fluid; and administering the reconstituted portion of the proteins to the mammalian subject. In further additional steps, the administering step includes administering the reconstituted portion of the proteins to the mammalian subject through a procedure selected from the group consisting of an oral administration, a rectal administration, a cutaneous administration, a subcutaneous administration, an intravenous injection, and an intramuscular injection.

In another embodiment, the component is selected from the group consisting of a liver component, a spleen component, a whole brain component, an ocular component, a gastro-intestinal component, a female specific component, and a male specific component.

In another aspect, the invention provides a composition. The composition includes a plurality of proteins from at least one lyophilized, blast frozen component of a harvested mammalian feto-placental unit. In one embodiment, the proteins include at least one essential fragment of at least one of the proteins. In another embodiment, the proteins include a plurality of chaperone proteins. In yet another embodiment, the lyophilized, blast frozen component is selected from the group consisting of a placenta-cord fetal component, a liver component, a spleen component, a whole brain component, an ocular component, a gastro-intestinal component, a female specific component, and a male specific component.

In an additional aspect, the invention provides a method of treatment of a disease or aging in a mammalian subject including administering to the mammalian subject a plurality of proteins from at least one blast frozen component harvested from a mammalian feto-placental unit; and reducing an accumulation of at least one intracellular protein in the mammalian subject. In one embodiment, the proteins include at least one essential fragment of at least one of the proteins. In another embodiment, the proteins include a plurality of chaperone proteins. In yet another embodiment, the step of reducing the accumulation of the intracellular protein comprises at least one of folding, refolding, degradation and transfer across a cellular membrane of the intracellular protein.

### BRIEF DESCRIPTION OF THE SEVERAL VIEWS OF THE DRAWINGS

Further features of the present invention will become apparent from the following description of embodiments thereof, given by way of example only, with reference to the accompanying drawings, in which:

FIG. 1 shows the steps of the method according to an embodiment of the invention;

FIG. 2 shows the steps of the method according to an embodiment of the invention;

FIG. 3 shows the steps of the method according to an embodiment of the invention;

FIG. 4 shows the steps of the method according to an embodiment of the invention;

FIG. 5 shows schematically the mis-folding and refolding of a protein, as described in the prior art;

FIG. 6 shows schematically the degradation of a protein by a chaperone protein, as described in the prior art;

FIG. 7 shows schematically cell apoptosis, as described in the prior art; and

FIG. 8 shows the steps of the method according to an embodiment of the invention;

### DETAILED DESCRIPTION OF THE INVENTION

This application claims priority to the previously filed U.S. Provisional Application No. 61/845,682 filed on Jul. 12, 2013 and hereby incorporates by reference that entire disclosure of Provisional Application No. 61/845,682.

The present invention relates to methods for harvesting proteins or essential fragments thereof, and in particular chaperone proteins, from the mammalian feto-placental unit, and the use of such mammalian feto-placental unit proteins in compositions and medical therapies for the treatment of disease and aging. For the purposes of the present application, the “essential fragment” of a protein of a mammalian feto-placental unit is defined as the portion of the protein which is capable of at least one of the reversal of cell apoptosis, the repair of cells, the regeneration of cells, and the regulation of protein folding, re-folding, transportation and degradation, through for example, the Ubiquitin Protease Pathway (UPP).

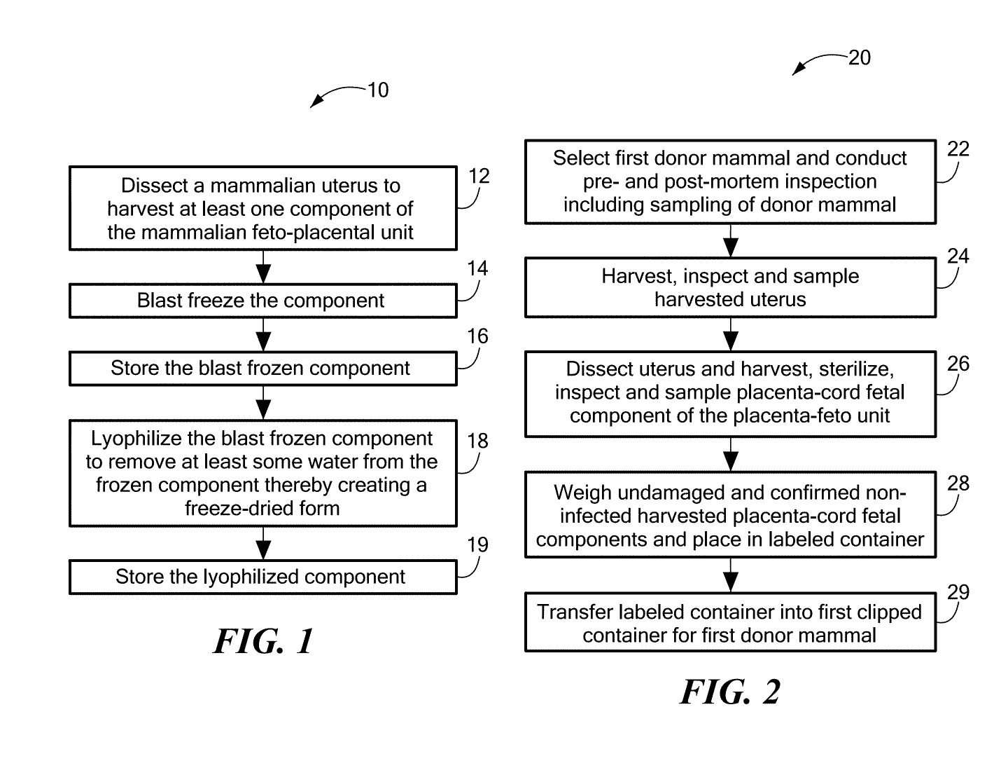

In one aspect, the invention provides a method 10 of harvesting and processing proteins from a mammalian feto-placental unit, as shown in FIG. 1. The method includes the steps of dissecting a mammalian uterus to harvest at least one component of the mammalian feto-placental unit, as shown in step 12; and blast freezing the component, as shown in step 14; wherein the component includes the proteins. In one embodiment, the method includes storing the blast frozen component, as shown in step 16. In another embodiment, the method includes lyophilizing the blast frozen component to remove at least some water from the frozen component thereby creating a freeze-dried form, as shown in step 18; and storing the lyophilized component, as shown in step 19.

In further embodiments, the invention provides the method 20 shown in FIG. 2. A female pregnant mammal, such as, for example, a pregnant ewe, is selected as a first donor mammal for supplying a uterus, as shown in step 22. A registered veterinarian conducts a pre-mortem and post-mortem examination of the selected first donor mammal to ensure that the donor is healthy and free of any visible disease. For example, the veterinarian inspects for muscular wasting which in an ovine could indicate parasitism or Johnes disease. The registered veterinarian also inspects for normal motor-function to rule out any central nervous system disease, such as, for example, aberrant larval migration, spinal cord and/or brain infection including infection in the middle and inner ear. The registered veterinarian also conducts a post-mortem examination of the selected first donor mammal to confirm the health and absence of disease in the donor mammal. The veterinarian can inspect for, for example, enlarged kidneys and nephrosis which could indicate a clostridial type infection. Core samples are taken from the liver and/or spleen of the first donor mammal for bacterial and viral isolation. For purposes of the present invention, only tissue negative to bacterial and viral isolation are harvested.

The uterus is harvested and inspected by the registered veterinarian to confirm the absence of lesions and/or other defects in the uterus, as shown in step 24. The uterus is dissected and the placenta with cotyledons and umbilical cord are separated from the remaining fetal mass and washed in a sterile non-pyrogenic solution containing no antimicrobial agents such as, for example, Lactated Ringers Solution (LRS), as shown in step 26. For the purposes of this application, the terms “placenta-cord fetal component or components” refer to the fetal placenta with cotyledons and umbilical cord which are harvested from the remaining fetal mass for a particular donor mammal while the term “feto-placental unit” includes the dissected placenta-cord fetal component plus the remaining dissected fetal mass. The harvested placenta-cord fetal components are inspected for damage such as, for example, tissue bruising or tearing which may have occurred during harvesting and dissection. Samples of the undamaged harvested placenta-cord fetal components are analyzed for infection using bacterial and/or viral cultures. Undamaged, confirmed non-infected harvested placenta-cord fetal components are weighed and placed in a labeled sterile sample container, as shown in step 28. The sterile sample container holding the placenta-cord fetal components is then transferred into a first clipped, that is, securely closed, sterile container, as shown in step 29 of FIG. 2. Damaged and/or infected dissected placenta-cord components are discarded.

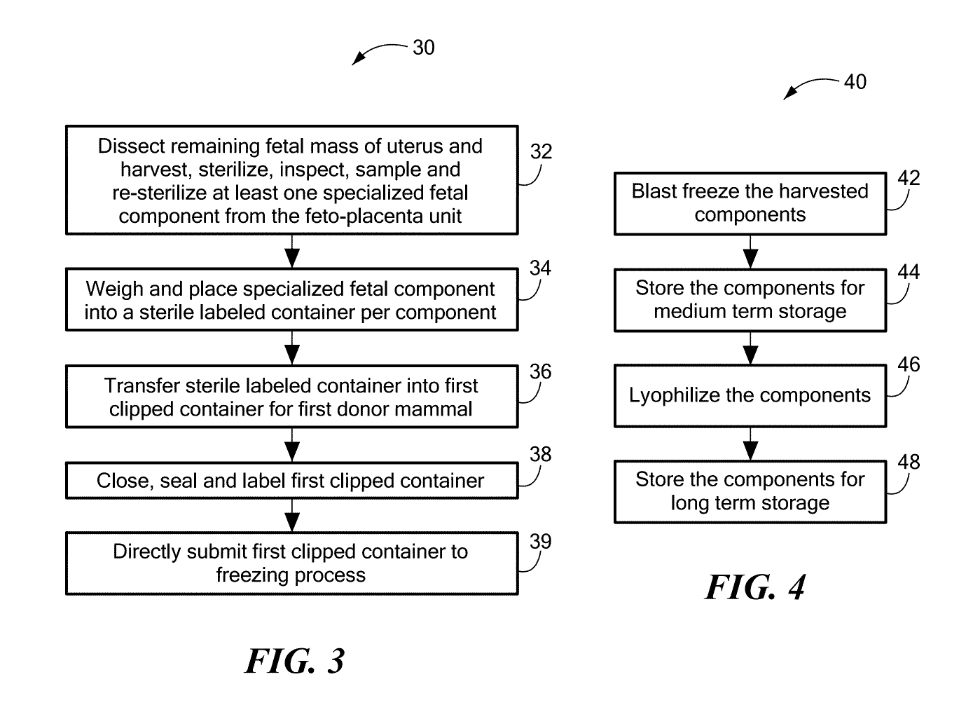

Further embodiments including the steps of method 30 are illustrated in FIG. 3. The remaining fetal mass not including the placenta-cord fetal components is also washed in a sterile non-pyrogenic solution containing no antimicrobial agents such as, for example, LRS, and transferred to a dissection table, as shown in step 32. Under sterile conditions, the sterilized remaining fetal mass is dissected into relevant specialized fetal components such as, for example, a liver component, a spleen component, a whole brain component, an ocular component, a gastro-intestinal component, a female specific component which includes accessory sexual glands, and a male specific component which includes male penile tissue, testicular tissue and accessory sexual glands. The harvested specialized fetal components are inspected for damage during dissection. Samples of undamaged harvested specialized fetal components are analyzed for infection using bacterial and/or viral cultures. Damaged and/or infected dissected specialized fetal components are discarded. Non-damaged, non-infected harvested specialized fetal components are again washed in a similar sterile, non-pyrogenic solution, such as, for example, LRS. Each re-sterilized harvested specialized fetal component is weighed and placed in its own sterile labeled container per component, as shown in step 34. The labeled containers holding the harvested specialized fetal components corresponding to the same first donor mammal are then transferred into the first clipped sterile container along with the placenta-cord fetal components for the same first donor mammal, as shown in step 36.

The first sterile clipped container is closed, sealed and labeled with identification information including weight and any notes regarding the dissection as shown in step 38. The prior multiple inspections of the entire feto-placental unit ensures that normal fetal growth has occurred and that there is no sign of infection and concomitant disease. The first sterile clipped container is directly submitted to a process for freezing the harvested components, as shown in step 39.

The freezing and storage processes 40 of the invention are shown in FIG. 4. The freezing process is accomplished using a fast or rapid freezing otherwise known as a blast freezing, as shown in step 42. The blast freezing lowers the temperature of the components to at least −6° C., and preferably to at least −10° C., and more preferably to at least −20° C., in eight hours, and preferably in six hours, and more preferably in four hours and most preferably in two hours.

After the blast freezing temperature is reached, the components can be stored in a freezer in medium term storage, as shown in step 44, for up to twelve months, and preferably for up to six months, and more preferably for up to three months, and most preferably for up to two months. In medium term storage, the temperature of the components is maintained between −20° C. and −100° C., and preferably between −40° C. and −90° C., and more preferably −60° C. and −80° C., and most preferably between −65° C. and −75° C.

Immediately following blast freezing, or following blast freezing and medium term storage as shown in step 44 of FIG. 4, the components can be further lyophilized or freeze dried to remove water from the components, as shown in step 46, using methods known to those of ordinary skill in the art. A freeze dried form of the component is thereby formed. Lyophilization is conducted on a batch basis by weight and according to type of component. Thus, the placenta-cord fetal component and the specialized liver, spleen, brain, ocular, gastro-intestinal, and female and male specific fetal components are lyophilized by weight according to component type to satisfy minimum weight requirements. Components are lyophilized to weigh a minimum of 10 grams of specific tissue, and preferably a minimum of 8 grams of specific tissue, and more preferably a minimum of 6 grams of specific tissue, and most preferably a minimum of 4 grams of specific tissue.

The components can then be stored for long term storage in their lyophilized, that is, freeze-dried form, as shown in step 48. The temperature of long term storage is maintained between 5° C. and 30° C., and preferably between 10° C. and 25° C., and more preferably between 15° C. and 20° C., and most preferably at 20° C. The duration of long term storage can equal as much as 100 years, and preferably a maximum of 50 years, and more preferably a maximum of 10 years, and even more preferably a maximum of 2 years, and most preferably a maximum of 1 year.

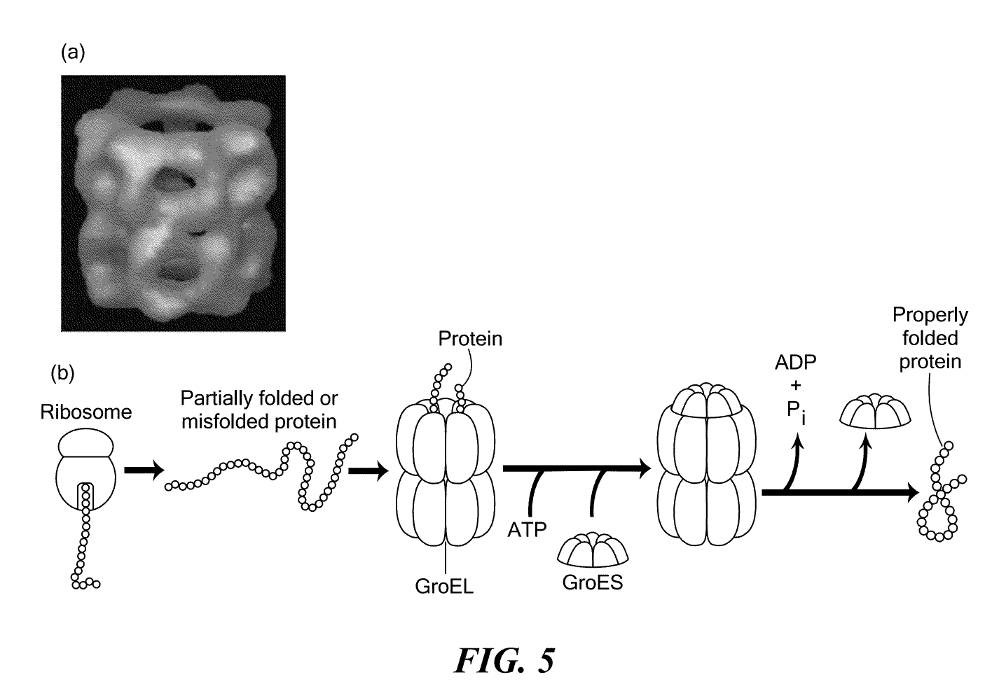

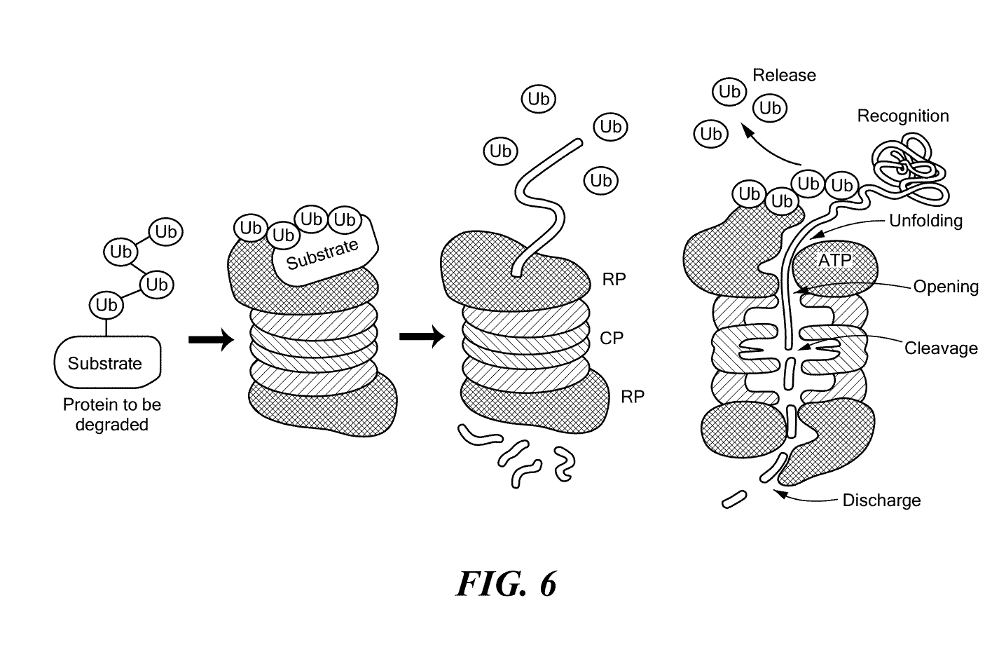

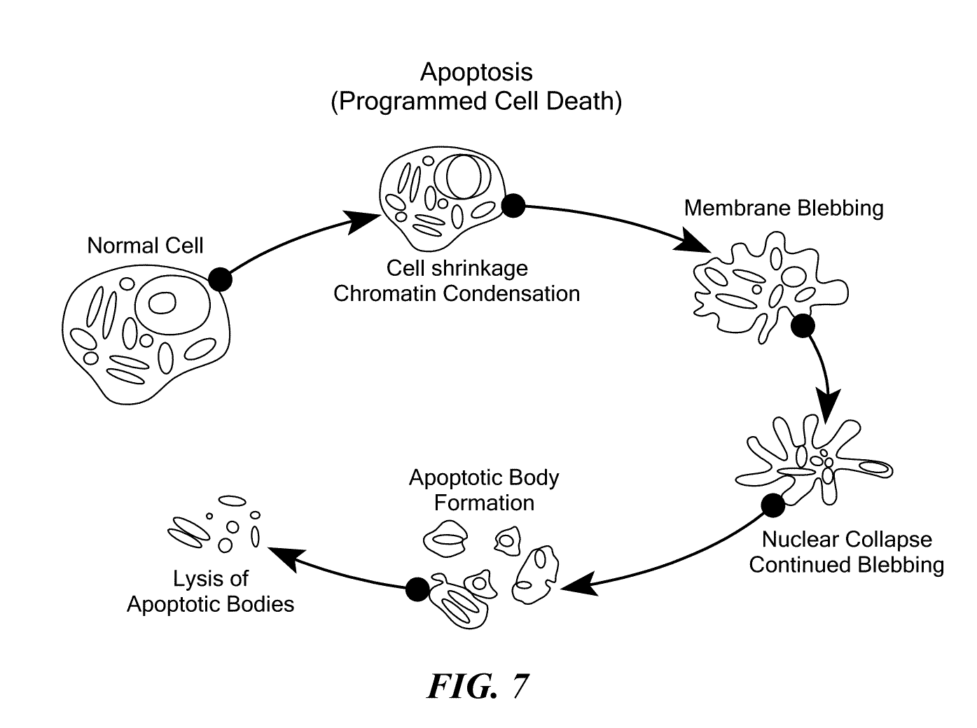

The harvested and processed mammalian fetal unit components include a plurality of proteins or essential fragments thereof. In one embodiment, the mammalian feto-placental unit protein or essential fragments include chaperone proteins. The chaperone proteins can have the ability to re-fold wrongly folded proteins as shown in FIG. 5. In addition or alternatively, the chaperone proteins can degrade otherwise not fully metabolized proteins. FIG. 6 demonstrates this mechanism, where UB identifies ubiquitors, CP identifies chaperone proteins, and RP identifies residual proteins. Furthermore, in addition or alternatively, the chaperone proteins can reverse apoptosis, as shown in FIG. 7. As a cell descends into the spiral of cell death, the cell most commonly goes through a biologic sequence of cellular shut down mechanisms. The shutdown mechanisms take various forms but in essence lead to intracellular protein accumulation mainly through a lack of protein transport out of the cell. The protein accumulation in turn leads to osmotic imbalance, cell membrane disruption and cell membrane disintegration. Chaperone proteins can work intracellularly to fold, refold, transport or degrade accumulated proteins. The reverse of toxic protein accumulation protein ameliorates osmotic imbalance thereby preventing membrane disruption. As a result, the cellular death spiral is reversed. Further, in addition or alternatively, the chaperone proteins can affect intracellular signaling by controlling conformational changes required for activation or deactivation of signaling proteins, and their assembly in specific signalosome complexes. See Tarone at abstract.

Accordingly, in embodiments of the method of the invention, the proteins of the components of the mammalian feto-placental unit can be used for medicinal purposes in the treatment of disease and/or aging in mammalian subjects including, for example, humans. Most disease and aging is characterized by some form of cellular dysfunction including the slowing of cellular mechanisms and the buildup of dysfunctional proteins within the cell. This cellular dysfunction leads to cellular under performance and clinical manifestation of disease and/or aging. The proteins of the components of the mammalian placental unit including essential fragments and chaperone proteins can be administered to promote folding, refolding, transport or degradation of accumulated proteins within cells.

An example of a disease mechanism which can be targeted by the method of the invention is the manifestation of type 2 diabetes. Although this disease can have multi-centric etiologies and manifestations, the cellular basis of the disease relates to the compromised ability of the cell to make insulin receptors and/or to respond to insulin/insulin receptor interaction because of the accumulation of proteins. The compromised cellular abilities lead to a lack of systemic glucose regulation which in turn leads to a cascade of multi-compartmental manifestations of clinical symptoms, but most specifically to an interference with arteriole circulation. The arteriole circulation interference leads to a decrease in blood circulation, vascular constriction, rise in blood pressure, and a decrease in vascular support for organs and cells. Ischemia, cellular apoptosis and eventually cell death result.

The method of the present invention including the administration of mammalian feto-placental unit proteins to a mammalian subject such as a human can counteract and at least partially reverse this cascade of events. In this example, the chaperone proteins administered to the human subject are from an exogenous biologic source of mammalian feto-placental tissue. After administration to the subject, the mammalian feto-placental unit proteins including chaperone proteins can work intracellularly to fold, refold, transport, and/or degrade accumulated proteins. These mechanisms at least partially reverse the toxic accumulation proteins. As a result, the cellular death spiral is reversed at least partially leading to the return of cellular function related to the response of the insulin/insulin receptor complex, the relaxation of arterioles, the decrease in blood pressure, the re oxygenation of tissue, the reverse of ischemia and the return to normal cell function and organ function.

At least portions of the tissues including the proteins can be extracted from the blast frozen components of the mammalian feto-placental unit, thawed and directly administered to the mammalian subject using different procedures employing, for example, a spatula, a sponge, a gel, and encapsulation. Non-limiting procedures for protein administration to a mammalian subject include a sublingual procedure, an intra-ocular procedure, an intra-rectal procedure and an intra-gastro-intestinal procedure.

Alternatively, at least portions of tissues including the proteins can be extracted from the lyophilized components of the mammalian feto-placental unit and reconstituted with a fluid, such as, for example, sterilized water or saline. The reconstituted proteins can be administered to the mammalian subject using various procedures such as, for non-limiting examples, an oral administration, a rectal administration, a cutaneous administration, a subcutaneous administration, an intravenous injection, and an intramuscular injection.

The proteins including the essential fragments thereof harvested from the mammalian feto-placental unit, processed and stored according to a method of the invention are included in the list provided in Table 1. All versions of the database: the uniprot-_{—}20130128_{—}5 wcYYr database are included for the purposes of the specification.

Examples of various diseases and/or symptoms characteristic of aging which can be treated by the proteins including the essential fragments thereof harvested from the mammalian feto-placental unit, and processed according to a method of the invention include, for example, type 2 diabetes, type 1 diabetes, cardiomyopathy, prostate cancer, renal dysfunction, and intra- and extra-cellular storage disease, Alzheimer disease, Parkinson disease, sexual dysfunction including female dysfunction (e.g., fluid reduction) and male dysfunction (e.g., erectile dysfunction), hormonal imbalance including hormonal imbalance around the cessation of mensus or menopause, musculoskeletal disorder, and neoplasia. For the purposes of this application, “intra- and extra-cellular storage disease” refers to conditions where the body makes proteins but does not have the mechanism such as, for example, the enzymes, to break down the proteins, and the accumulation of such proteins becomes harmful for the subject. Selected proteins are known to those of ordinary skill in the art to ammeliorate selected specific diseases as discussed in, for example, Growth charts for patients with Hunter syndrome, Patel P, Suzuki Y, Maeda M, Yasuda E, Shimada T, Orii K E, Orii T, Tomatsu S., Mol Genet Metab Rep. 2014; 1:5-18, PMID: 24955330, [PubMed], and other articles identified in http://www.ncbi.nlm.nih.gov/pubmed/?term=mucopolysaccharide+storage+disease.

In another aspect, the invention provides a composition including proteins originating from at least one blast frozen, lyophilized component of a harvested mammalian feto-placental unit. In one embodiment, the proteins include at least one essential fragment of at least one of the proteins. In another embodiment, the proteins include chaperone proteins. In yet other embodiments, the component is selected from the group consisting of at least one of a placenta-cord fetal component, a liver component, a spleen component, a whole brain component, an ocular component, a gastro-intestinal component, a female specific component, and a male specific component.

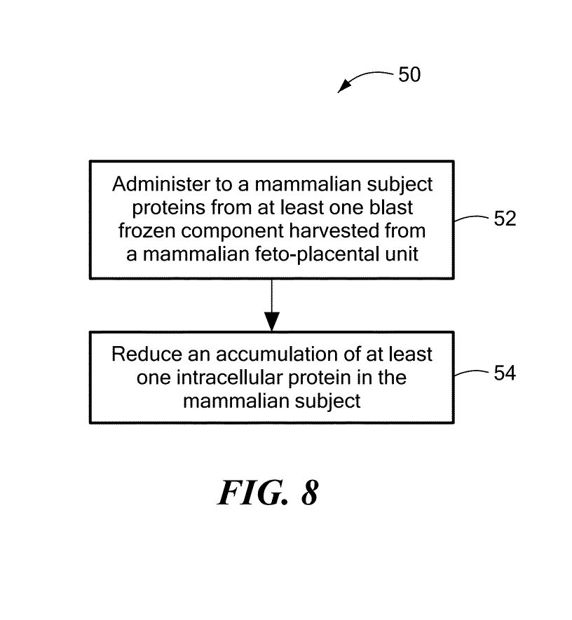

An additional aspect of the invention is shown by method 50 in FIG. 8 which provides for the treatment of a disease or aging in a mammalian subject. The method includes administering to the mammalian subject a plurality of proteins from at least one blast frozen component harvested from a mammalian feto-placental unit, as shown in step 52; and reducing an accumulation of at least one intracellular protein in the mammalian subject, as shown in step 54. In one embodiment, the step of reducing the accumulation of the intracellular protein includes at least one of folding, refolding, transfer, and degradation of the intracellular protein.

### Example

A donor sheep certified according to New Zealand Protocol was selected. Placenta-cord, liver, gastro-intestinal and specific male fetal components were dissected, harvested, and processed including inspection, sterilization, blast freezing followed by lyophilization. The lyophilized components were stored for at least of two years. Tissue samples were then extracted from the lyophilized components periodically according to the patient treatment protocol and reconstituted with sterilized saline to form 6 ml injection samples including 1 gram of reconstituted lyophilized component per each sample.

At the onset of treatment, Patient X was in his early 90's and had concomitant pro static cancer which exhibited as an enlargement of the prostate gland coupled with a prostate specific antigen (PSA) score of over 20. The patient had experienced baldness for approximately 40 years. The patient suffered from clinical stiffness and exhibited stooped posture due to osteoporosis. The patient reported experiencing erectile dysfunction and low libido.

The injection samples including the lyophilized components reconstituted with sterilized saline were injected subcutaneously into Patient X once per month for 6 months and once per 3 months thereafter for 5 years.

After two months of treatment, the patient's clinical physician observed a PSA of 15 and a decrease in prostatic gland size to 30% of the pre-treatment size. Subsequently, the patient's PSA dropped to less than 5 and remained less than 5 thereafter. New hair growth was observed on top of the patient's head. The patient no longer suffered from pre-treatment clinical stiffness and maintained an erect posture. The patient reported return of libido and erectile function.

The foregoing examples and detailed description are not to be deemed limiting of the invention which is defined by the following claims. The invention is understood to encompass such obvious modifications thereof as would be apparent to those of ordinary skill in the art.

TABLE 1

| | | Version: Unspecified | | | | | |
|---|---|---|---|---|---|---|---|
| | | Charge States | | | | | |
| | | Calculated: unknown | | | | | |
| | | Deisotoped: unknown | | | | | |
| | | Textual Annotation: | | | | | |
| | Database Set: 1 | | | | | | |
| | Database | | | | | | |
| | | Database Name: the | | | | | |
| | | uniprot-_20130128_5wcYYr | | | | | |
| | | database | | | | | |
| | | | Version: unknown | | | | |
| | | | Taxonomy: All Entries | | | | |
| | | | Number of Proteins: | | | | |
| | | | 52864 | | | | |
| | | Does database contain | | | | | |
| | | common contaminants?: | | | | | |
| | | unknown | | | | | |
| | Search Engine | | | | | | |
| | Set: 1 | | | | | | |
| | Search Engine | | | | | | |
| | | Search Engine: | | | | | |
| | | X! Tandem | | | | | |
| | | | Version: TORNADO | | | | |
| | | | (2010.01.01.4) | | | | |
| | | | Samples: All Samples | | | | |
| | | | Fragment Tolerance: | | | | |
| | | | 20 PPM (Monoisotopic) | | | | |
| | | | Parent Tolerance: | | | | |
| | | | 20 PPM (Monoisotopic) | | | | |
| | | | Fixed Modifications: | | | | |
| | | | +57 on C (Carbamidomethyl) | | | | |
| | | | Variable Modifications: | | | | |
| | | | +1 on NQ (Deamidation), | | | | |
| | | | +16 on MW (Oxidation), | | | | |
| | | | +32 on M (Sulphone), | | | | |
| | | | +32 on W (formylkynurenin), | | | | |
| | | | +42 on n (Acetyl) | | | | |
| | | | Database: | | | | |
| | | | the uniprot-_20130128_5wcYYr | | | | |
| | | | database (unknown version, | | | | |
| | | | 52864 entries) | | | | |
| | | | Digestion Enzyme: | | | | |
| | | | Max Missed Cleavages: 1 | | | | |
| | Scaffold Version: | | | | | | |
| | Scaffold_3.6.5 | | | | | | |
| | | | MaxQuant: Andromeda P-score of | | | | |
| | | | greater than 50, Sequest: deltaCn | | | | |
| | | | scores of greater than 0.10 and | | | | |
| | | | XCorr scores of greater than 1.8, | | | | |
| | | | 2.5, 3.5 and 3.5 for singly, doubly, | | | | |
| | | | triply and quadruply charged peptides, | | | | |
| | | | ZCore Proteome Discoverer: ZCore scores | | | | |
| | | | of greater than 100, Mascot: ion | | | | |
| | | | scores must be greater than both the | | | | |
| | | | associated | | | | |
| | | | Protein Thresholds: 1 peptides minimum | | | | |
| | | | | | | | forward |
| | | | | | | Protein | |
| | | | | | Molecular | Grouping | qe01225013_{—} |
| Number | Visible? | Starred? | Identified Proteins (516) | Accession Number | Weight | Ambiguity | 6_stanley.mgf |
| 1 | TRUE | FALSE | Uncharacterized protein | tr \| F7AAK7 \| | 42 kDa | TRUE | 485 |
| | | | OS = Equus caballus | F7AAK7_HORSE | | | |
| | | | GN = ACTG1 PE = 3 SV = 1 | | | | |
| 2 | TRUE | FALSE | sp \| ALBU_BOVIN \| | sp \| ALBU_BOVIN \| | 69 kDa | TRUE | 216 |
| 3 | TRUE | FALSE | Uncharacterized protein | tr \| F6XTY0 \| | 206 kDa | TRUE | 197 |
| | | | (Fragment) | F6XTY0_HORSE | | | |
| | | | OS = Equus caballus | | | | |
| | | | GN = MYH9 PE = 4 SV = 1 | | | | |
| 4 | TRUE | FALSE | Tropomyosin alpha-4 chain | sp \| P02561 \| | 29 kDa | TRUE | 213 |
| | | | OS = Equus caballus | TPM4_HORSE | | | |
| | | | GN = TPM4 PE = 1 SV = 2 | | | | |
| 5 | TRUE | FALSE | Uncharacterized protein | tr \| F7B5C4 \| | 42 kDa | TRUE | 186 |
| | | | (Fragment) | F7B5C4_HORSE | | | |
| | | | OS = Equus caballus | | | | |
| | | | GN = VIM PE = 3 SV = 1 | | | | |
| 6 | TRUE | FALSE | Hemoglobin subunit beta | sp \| P67823 \| | 16 kDa | TRUE | 140 |
| | | | OS = Equus hemionus kulan | HBB_EQUHE (+1) | | | |
| | | | GN = HBB PE = 1 SV = 1 | | | | |
| 7 | TRUE | FALSE | Histone H2B | tr \| F6TWM4 \| | 14 kDa | TRUE | 115 |
| | | | OS = Equus caballus | F6TWM4_HORSE | | | |
| | | | GN = HIST1H2BK PE = 3 SV = 1 | | | | |
| 8 | TRUE | FALSE | Annexin | tr \| F6ZI51 \| | 39 kDa | | 115 |
| | | | OS = Equus caballus | F6ZI51_HORSE | | | |
| | | | GN = ANXA2 PE = 3 SV = 1 | | | | |
| 9 | TRUE | FALSE | 78 kDa glucose-regulated protein | tr \| F6YG82 \| | 72 kDa | TRUE | 96 |
| | | | (Fragment) | F6YG82_HORSE | | | |
| | | | OS = Equus caballus | | | | |
| | | | GN = HSPA5 PE = 3 SV = 1 | | | | |
| 10 | TRUE | FALSE | Heat shock cognate 71 kDa protein | sp \| A2Q0Z1 \| | 71 kDa | TRUE | 91 |
| | | | OS = Equus caballus | HSP7C_HORSE | | | |
| | | | GN = HSPA8 PE = 2 SV = 1 | (+1) | | | |
| 11 | TRUE | FALSE | | tr \| F7BPT4 \| | 69 kDa | TRUE | 73 |
| | | | | F7BPT4_HORSE | | | |
| 12 | TRUE | FALSE | | sp \| HBA_HUMAN \| | 15 kDa | TRUE | 68 |
| 13 | TRUE | FALSE | Uncharacterized protein | tr \| F6RGN2 \| | 15 kDa | | 80 |
| | | | OS = Equus caballus | F6RGN2_HORSE | | | |
| | | | GN = FABP5 PE = 3 SV = 1 | | | | |
| 14 | TRUE | FALSE | Uncharacterized protein | tr \| F7A281 \| | 33 kDa | TRUE | 70 |
| | | | OS = Equus caballus | F7A281_HORSE | | | |
| | | | GN = TPM2 PE = 3 SV = 1 | | | | |
| 15 | TRUE | FALSE | Uncharacterized protein | tr \| F6ZUJ2 \| | 92 kDa | TRUE | 65 |
| | | | OS = Equus caballus | F6ZUJ2_HORSE | | | |
| | | | GN = GRP94 PE = 3 SV = 1 | | | | |
| 16 | TRUE | FALSE | Uncharacterized protein | tr \| F6VSN9 \| | 57 kDa | | 63 |
| | | | (Fragment) | F6VSN9_HORSE | | | |
| | | | OS = Equus caballus | | | | |
| | | | GN = PDIA3 PE = 3 SV = 1 | | | | |
| 17 | TRUE | FALSE | Uncharacterized protein | tr \| F7DXG8 \| | 19 kDa | TRUE | 64 |
| | | | OS = Equus caballus | F7DXG8_HORSE | | | |
| | | | GN = CFL1 PE = 4 SV = 1 | | | | |
| 18 | TRUE | FALSE | Uncharacterized protein | tr \| F6ZY40 \| | 74 kDa | TRUE | 65 |
| | | | OS = Equus caballus | F6ZY40_HORSE | | | |
| | | | GN = LMNA PE = 3 SV = 1 | | | | |
| 19 | TRUE | FALSE | Uncharacterized protein | tr \| F6YV53 \| | 87 kDa | TRUE | 53 |
| | | | (Fragment) | F6YV53_HORSE | | | |
| | | | OS = Equus caballus | | | | |
| | | | GN = ACTN4 PE = 4 SV = 1 | | | | |
| 20 | TRUE | FALSE | Uncharacterized protein | tr \| F7B821 \| | 52 kDa | | 57 |
| | | | (Fragment) | F7B821_HORSE | | | |
| | | | OS = Equus caballus | | | | |
| | | | GN = P4HB PE = 3 SV = 1 | | | | |
| 21 | TRUE | FALSE | Histone H3 | tr \| F6Y7V5 \| | 15 kDa | TRUE | 42 |
| | | | OS = Equus caballus | F6Y7V5_HORSE | | | |
| | | | GN = LOC100147089 PE = 3 SV = 1 | | | | |
| 22 | TRUE | FALSE | Uncharacterized protein | tr \| F7BV56 \| | 211 kDa | TRUE | 46 |
| | | | OS = Equus caballus | F7BV56_HORSE | | | |
| | | | GN = MYH14 PE = 4 SV = 1 | | | | |
| 23 | TRUE | FALSE | Uncharacterized protein | tr \| F6WPZ3 \| | 503 kDa | | 46 |
| | | | OS = Equus caballus | F6WPZ3_HORSE | | | |
| | | | GN = PLEC PE = 4 SV = 1 | (+5) | | | |
| 24 | TRUE | FALSE | Uncharacterized protein | tr \| F6S2F8 \| | 21 kDa | | 47 |
| | | | OS = Equus caballus | F6S2F8_HORSE | | | |
| | | | GN = HIST1H1C PE = 3 SV = 1 | | | | |
| 25 | TRUE | FALSE | Uncharacterized protein | tr \| F6TQR2 \| | 48 kDa | | 41 |
| | | | OS = Equus caballus | F6TQR2_HORSE | | | |
| | | | GN = CALR PE = 3 SV = 1 | | | | |
| 26 | TRUE | FALSE | Enolase 1 | tr \| F6V7C1 \| | 47 kDa | | 38 |
| | | | OS = Equus caballus | F6V7C1_HORSE | | | |
| | | | GN = ENO1 PE = 2 SV = 1 | | | | |
| 27 | TRUE | FALSE | Heat shock protein HSP 90-alpha | sp \| Q9GKX7 \| | 85 kDa | TRUE | 39 |
| | | | OS = Equus caballus | HS90A_HORSE | | | |
| | | | GN = HSP90AA1 | | | | |
| 28 | TRUE | FALSE | Uncharacterized protein | tr \| F6XEB4 \| | 28 kDa | TRUE | 40 |
| | | | OS = Equus caballus | F6XEB4_HORSE | | | |
| | | | GN = YWHAZ PE = 3 SV = 1 | | | | |
| 29 | TRUE | FALSE | sp \| DHE3_BOVIN \| | sp \| DHE3_BOVIN \| | 62 kDa | | 39 |
| 30 | TRUE | FALSE | Uncharacterized protein | tr \| F7BGS8 \| | 280 kDa | TRUE | 33 |
| | | | OS = Equus caballus | F7BGS8_HORSE | | | |
| | | | GN = FLNA PE = 4 SV = 1 | (+1) | | | |
| 31 | TRUE | FALSE | Uncharacterized protein | tr \| F7DZ82 \| | 99 kDa | TRUE | 39 |
| | | | (Fragment) | F7DZ82_HORSE | | | |
| | | | OS = Equus caballus | (+1) | | | |
| | | | GN = ACTN1 PE = 4 SV = 1 | | | | |
| 32 | TRUE | FALSE | sp \| GELS_HUMAN \| | sp \| GELS_HUMAN \| | 86 kDa | TRUE | 39 |
| 33 | TRUE | FALSE | Uncharacterized protein | tr \| F7BWW6 \| | 89 kDa | | 36 |
| | | | (Fragment) | F7BWW6_HORSE | | | |
| | | | OS = Equus caballus | | | | |
| | | | GN = VCP PE = 3 SV = 1 | | | | |
| 34 | TRUE | FALSE | Uncharacterized protein | tr \| F7DMQ3 \| | 36 kDa | | 32 |
| | | | OS = Equus caballus | F7DMQ3_HORSE | | | |
| | | | GN = AKR1B1 PE = 4 SV = 1 | | | | |
| 35 | TRUE | FALSE | Uncharacterized protein | tr \| F6WA57 \| | 70 kDa | TRUE | 32 |
| | | | (Fragment) | F6WA57_HORSE | | | |
| | | | OS = Equus caballus | | | | |
| | | | GN = LCP1 PE = 4 SV = 1 | | | | |
| 36 | TRUE | FALSE | Elongation factor 1-alpha 1 | sp \| A2Q0Z0 \| | 50 kDa | | 29 |
| | | | OS = Equus caballus | EF1A1_HORSE | | | |
| | | | GN = EEF1A1 PE = 2 SV = 1 | (+1) | | | |
| 37 | TRUE | FALSE | Triosephosphate isomerase | tr \| F6TZS9 \| | 27 kDa | | 35 |
| | | | OS = Equus caballus | F6TZS9_HORSE | | | |
| | | | GN = TPI1 PE = 3 SV = 1 | | | | |
| 38 | TRUE | FALSE | Glyceraldehyde-3-phosphate | tr \| F6YV40 \| | 36 kDa | TRUE | 33 |
| | | | dehydrogenase | F6YV40_HORSE | | | |
| | | | OS = Equus caballus | | | | |
| | | | GN = LOC100054877 PE = 3 | | | | |
| 39 | TRUE | FALSE | Uncharacterized protein | tr \| F7AX54 \| | 17 kDa | | 35 |
| | | | OS = Equus caballus | F7AX54_HORSE | | | |
| | | | GN = MYL6 PE = 4 SV = 1 | | | | |
| 40 | TRUE | FALSE | sp \| PPIA_HUMAN \| | sp \| PPIA_HUMAN \| | 18 kDa | TRUE | 34 |
| 41 | TRUE | FALSE | Pyruvate kinase | tr \| B3IVM0 \| | 58 kDa | | 28 |
| | | | OS = Equus caballus | B3IVM0_HORSE | | | |
| | | | GN = PKM PE = 2 SV = 1 | (+2) | | | |
| 42 | TRUE | FALSE | Uncharacterized protein | tr \| F7CS04 \| | 29 kDa | TRUE | 34 |
| | | | (Fragment) | F7CS04_HORSE | | | |
| | | | OS = Equus caballus | | | | |
| | | | GN = TPM1 PE = 3 SV = 1 | | | | |
| 43 | TRUE | FALSE | Fructose-bisphosphate aldolase | tr \| F6SX98 \| | 41 kDa | | 30 |
| | | | (Fragment) | F6SX98_HORSE | | | |
| | | | OS = Equus caballus | | | | |
| | | | GN = ALDOA PE = 3 SV = 1 | | | | |
| 44 | TRUE | FALSE | Uncharacterized protein | tr \| F7E0H3 \| | 50 kDa | TRUE | 27 |
| | | | OS = Equus caballus | F7E0H3_HORSE | | | |
| | | | GN = TUBB PE = 3 SV = 1 | | | | |
| 45 | TRUE | FALSE | Uncharacterized protein | tr \| F6UUS3 \| | 95 kDa | TRUE | 30 |
| | | | (Fragment) | F6UUS3_HORSE | | | |
| | | | OS = Equus caballus | | | | |
| | | | GN = EEF2 PE = 4 SV = 1 | | | | |
| 46 | TRUE | FALSE | Uncharacterized protein | tr \| F6ZXQ2 \| | 43 kDa | TRUE | 29 |
| | | | OS = Equus caballus | F6ZXQ2_HORSE | | | |
| | | | GN = LOC100147127 PE = 3 SV = 1 | | | | |
| 47 | TRUE | FALSE | Uncharacterized protein | tr \| F7BV85 \| | 193 kDa | | 25 |
| | | | (Fragment) | F7BV85_HORSE | | | |
| | | | OS = Equus caballus | | | | |
| | | | GN = CLTC PE = 4 SV = 1 | | | | |
| 48 | TRUE | FALSE | Uncharacterized protein | tr \| F7CQA4 \| | 173 kDa | | 26 |
| | | | OS = Equus caballus | F7CQA4_HORSE | | | |
| | | | GN = LAMC1 PE = 4 SV = 1 | (+1) | | | |
| 49 | TRUE | FALSE | ATP synthase subunit beta | tr \| F6U187 \| | 56 kDa | | 26 |
| | | | OS = Equus caballus | F6U187_HORSE | | | |
| | | | GN = ATP5B PE = 3 SV = 1 | | | | |
| 50 | TRUE | FALSE | Uncharacterized protein | tr \| F6VSC4 \| | 228 kDa | TRUE | 15 |
| | | | OS = Equus caballus | F6VSC4_HORSE | | | |
| | | | GN = MYH11 PE = 4 SV = 1 | (+3) | | | |
| 51 | TRUE | FALSE | Histone H4 | tr \| F6VFV9 \| | 11 kDa | | 24 |
| | | | OS = Equus caballus | F6VFV9_HORSE | | | |
| | | | GN = HIST2H4A PE = 3 SV = 1 | | | | |
| 52 | TRUE | FALSE | Uncharacterized protein | tr \| F6XT77 \| | 267 kDa | TRUE | 22 |
| | | | (Fragment) | F6XT77_HORSE | | | |
| | | | OS = Equus caballus | | | | |
| | | | GN = FLNB PE = 4 SV = 1 | | | | |
| 53 | TRUE | FALSE | High mobility group protein B1 | sp \| Q08IE6 \| | 25 kDa | TRUE | 23 |
| | | | OS = Equus caballus | HMGB1_HORSE | | | |
| | | | GN = HMGB1 PE = 2 SV = 3 | (+1) | | | |
| 54 | TRUE | FALSE | Lactate dehydrogenase B | tr \| B7XH74 \| | 37 kDa | | 19 |
| | | | OS = Equus caballus | B7XH74_HORSE | | | |
| | | | GN = LDHB PE = 2 SV = 1 | (+1) | | | |
| 55 | TRUE | FALSE | Uncharacterized protein | tr \| F6ZSB4 \| | 50 kDa | | 21 |
| | | | OS = Equus caballus | F6ZSB4_HORSE | | | |
| | | | GN = TUBA1A PE = 3 SV = 1 | (+1) | | | |
| 56 | TRUE | FALSE | Heat shock protein HSP 90-beta | sp \| Q9GKX8 \| | 83 kDa | TRUE | 23 |
| | | | OS = Equus caballus | HS90B_HORSE | | | |
| | | | GN = HSP90AB1 | | | | |
| 57 | TRUE | FALSE | Uncharacterized protein | tr \| F6YZ44 \| | 39 kDa | | 24 |
| | | | OS = Equus caballus | F6YZ44_HORSE | | | |
| | | | GN = HNRNPA1 PE = 4 SV = 1 | | | | |
| 58 | TRUE | FALSE | Uncharacterized protein | tr \| F6Z8K0 \| | 42 kDa | TRUE | 17 |
| | | | OS = Equus caballus | F6Z8K0_HORSE | | | |
| | | | GN = ACTG2 PE = 3 SV = 1 | | | | |
| 59 | TRUE | FALSE | Actin, cytoplasmic 1 | sp \| P60708 \| | 42 kDa | TRUE | 23 |
| | | | OS = Equus caballus | ACTB_HORSE | | | |
| | | | GN = ACTB PE = 2 SV = 1 | | | | |
| 60 | TRUE | FALSE | Uncharacterized protein | tr \| F7C0I7 \| | 466 kDa | | 17 |
| | | | (Fragment) | F7C0I7_HORSE | | | |
| | | | OS = Equus caballus | (+1) | | | |
| | | | GN = HSPG2 PE = 4 SV = 1 | | | | |
| 61 | TRUE | FALSE | Uncharacterized protein | tr \| F6ZTR4 \| | 33 kDa | TRUE | 22 |
| | | | OS = Equus caballus | F6ZTR4_HORSE | | | |
| | | | GN = TPM3 PE = 3 SV = 1 | | | | |
| 62 | TRUE | FALSE | Histone H2A | tr \| F6W1T4 \| | 14 kDa | TRUE | 20 |
| | | | OS = Equus caballus | F6W1T4_HORSE | | | |
| | | | GN = LOC100063021 PE = 3 SV = 1 | (+5) | | | |
| 63 | TRUE | FALSE | Uncharacterized protein | tr \| F6ZSZ5 \| | 117 kDa | | 22 |
| | | | OS = Equus caballus | F6ZSZ5_HORSE | | | |
| | | | GN = VCL PE = 4 SV = 1 | (+2) | | | |
| 64 | TRUE | FALSE | Uncharacterized protein | tr \| F7CBT8 \| | 54 kDa | | 18 |
| | | | (Fragment) | F7CBT8_HORSE | | | |
| | | | OS = Equus caballus | | | | |
| | | | GN = SLC2A1 PE = 3 SV = 1 | | | | |
| 65 | TRUE | FALSE | Serotransferrin | sp \| P27425 \| | 78 kDa | TRUE | 16 |
| | | | OS = Equus caballus | TRFE_HORSE | | | |
| | | | GN = TF PE = 2 | (+1) | | | |
| 66 | TRUE | FALSE | Uncharacterized protein | tr \| F7DW69 \| | 70 kDa | TRUE | 8 |
| | | | (Fragment) | F7DW69_HORSE | | | |
| | | | OS = Equus caballus | | | | |
| | | | GN = HSPA1A PE = 3 SV = 1 | | | | |
| 67 | TRUE | FALSE | Isocitrate dehydrogenase [NADP] | tr \| F6R7J2 \| | 48 kDa | | 19 |
| | | | (Fragment) | F6R7J2_HORSE | | | |
| | | | OS = Equus caballus | | | | |
| | | | GN = IDH1 PE = 3 SV = 1 | | | | |
| 68 | TRUE | FALSE | Uncharacterized protein | tr \| F6T0A6 \| | 11 kDa | | 19 |
| | | | (Fragment) | F6T0A6_HORSE | | | |
| | | | OS = Equus caballus | | | | |
| | | | GN = HSPE1 PE = 3 SV = 1 | | | | |
| 69 | TRUE | FALSE | Uncharacterized protein | tr \| F6Y014 \| | 31 kDa | | 18 |
| | | | (Fragment) | F6Y014_HORSE | | | |
| | | | OS = Equus caballus | | | | |
| | | | GN = CAPZB PE = 4 SV = 1 | | | | |
| 70 | TRUE | FALSE | Uncharacterized protein | tr \| F6W354 \| | 46 kDa | | 19 |
| | | | (Fragment) | F6W354_HORSE | | | |
| | | | OS = Equus caballus | | | | |
| | | | GN = ACTR3 PE = 3 SV = 1 | | | | |
| 71 | TRUE | FALSE | Uncharacterized protein | tr \| F7BND9 \| | 11 kDa | | 15 |
| | | | (Fragment) | F7BND9_HORSE | | | |
| | | | OS = Equus caballus | | | | |
| | | | GN = S100A11 PE = 4 SV = 1 | | | | |
| 72 | TRUE | FALSE | Uncharacterized protein | tr \| F6WKL7 \| | 55 kDa | TRUE | 18 |
| | | | OS = Equus caballus | F6WKL7_HORSE | | | |
| | | | PE = 3 SV = 1 | (+1) | | | |
| 73 | TRUE | FALSE | Uncharacterized protein | tr \| F7AQ43 \| | 53 kDa | TRUE | 16 |
| | | | OS = Equus caballus | F7AQ43_HORSE | | | |
| | | | GN = DES PE = 3 SV = 1 | | | | |
| 74 | TRUE | FALSE | Uncharacterized protein | tr \| F6PUX2 \| | 68 kDa | TRUE | 12 |
| | | | OS = Equus caballus | F6PUX2_HORSE | | | |
| | | | GN = MSN PE = 4 SV = 1 | | | | |
| 75 | TRUE | FALSE | Uncharacterized protein | tr \| F7B9Q9 \| | 55 kDa | | 16 |
| | | | OS = Equus caballus | F7B9Q9_HORSE | | | |
| | | | GN = PACSIN2 PE = 4 SV = 1 | | | | |
| 76 | TRUE | FALSE | Coronin (Fragment) | tr \| F7DEW5 \| | 53 kDa | TRUE | 15 |
| | | | OS = Equus caballus | F7DEW5_HORSE | | | |
| | | | GN = CORO1C PE = 3 SV = 1 | | | | |
| 77 | TRUE | FALSE | Uncharacterized protein | tr \| F6SXQ9 \| | 21 kDa | TRUE | 17 |
| | | | OS = Equus caballus | F6SXQ9_HORSE | | | |
| | | | GN = CDC42 PE = 3 SV = 1 | | | | |
| 78 | TRUE | FALSE | Malate dehydrogenase | tr \| F6TYW4 \| | 36 kDa | | 15 |
| | | | OS = Equus caballus | F6TYW4_HORSE | | | |
| | | | GN = MDH2 PE = 3 SV = 1 | | | | |
| 79 | TRUE | FALSE | Uncharacterized protein | tr \| F7E4G6 \| | 67 kDa | | 14 |
| | | | (Fragment) | F7E4G6_HORSE | | | |
| | | | OS = Equus caballus | | | | |
| | | | GN = HNRNPU PE = 4 SV = 1 | | | | |
| 80 | TRUE | FALSE | Uncharacterized protein | tr \| F7ALF9 \| | 67 kDa | TRUE | 13 |
| | | | (Fragment) | F7ALF9_HORSE | | | |
| | | | OS = Equus caballus | | | | |
| | | | GN = LMNB2 PE = 3 SV = 1 | | | | |
| 81 | TRUE | FALSE | Uncharacterized protein | tr \| F6WHS9 \| | 194 kDa | | 15 |
| | | | (Fragment) | F6WHS9_HORSE | | | |
| | | | OS = Equus caballus | (+3) | | | |
| | | | GN = LAMB1 PE = 4 SV = 1 | | | | |
| 82 | TRUE | FALSE | Uncharacterized protein | tr \| F7D9N9 \| | 61 kDa | | 12 |
| | | | OS = Equus caballus | F7D9N9_HORSE | | | |
| | | | GN = CALD1 PE = 4 SV = 1 | | | | |
| 83 | TRUE | FALSE | Uncharacterized protein | tr \| F6VY00 \| | 42 kDa | TRUE | 11 |
| | | | OS = Equus caballus | F6VY00_HORSE | | | |
| | | | GN = LOC100058814 PE = 3 SV = 1 | | | | |
| 84 | TRUE | FALSE | Uncharacterized protein | tr \| F6SP02 \| | 28 kDa | TRUE | 16 |
| | | | OS = Equus caballus | F6SP02_HORSE | | | |
| | | | GN = YWHAQ PE = 3 SV = 1 | | | | |
| 85 | TRUE | FALSE | Gelsolin | sp \| Q28372 \| | 81 kDa | TRUE | 14 |
| | | | OS = Equus caballus | GELS_HORSE | | | |
| | | | GN = GSN PE = 1 SV = 2 | | | | |
| 86 | TRUE | FALSE | Uncharacterized protein | tr \| F6PZ47 \| | 102 kDa | | 15 |
| | | | OS = Equus caballus | F6PZ47_HORSE | | | |
| | | | GN = SND1 PE = 4 SV = 1 | | | | |
| 87 | TRUE | FALSE | Uncharacterized protein | tr \| F6Q4Q1 \| | 51 kDa | | 14 |
| | | | OS = Equus caballus | F6Q4Q1_HORSE | | | |
| | | | GN = HNRNPK PE = 4 SV = 1 | | | | |
| 88 | TRUE | FALSE | Metalloproteinase inhibitor 2 | tr \| F7BEE7 \| | 20 kDa | | 13 |
| | | | (Fragment) | F7BEE7_HORSE | | | |
| | | | OS = Equus caballus | | | | |
| | | | GN = TIMP2 PE = 4 SV = 1 | | | | |
| 89 | TRUE | FALSE | Uncharacterized protein | tr \| F7CLY2 \| | 104 kDa | | 7 |
| | | | OS = Equus caballus | F7CLY2_HORSE | | | |
| | | | GN = AP1B1 PE = 4 SV = 1 | (+1) | | | |
| 90 | TRUE | FALSE | Phosphoglycerate kinase 1 | sp \| P00559 \| | 45 kDa | | 12 |
| | | | OS = Equus caballus | PGK1_HORSE | | | |
| | | | GN = PGK1 PE = 1 SV = 2 | | | | |
| 91 | TRUE | FALSE | Uncharacterized protein | tr \| F7DJS6 \| | 38 kDa | | 14 |
| | | | OS = Equus caballus | F7DJS6_HORSE | | | |
| | | | GN = GALM PE = 4 SV = 1 | | | | |
| 92 | TRUE | FALSE | sp \| CATD_HUMAN \| | sp \| CATD_HUMAN \| | 45 kDa | | 14 |
| 93 | TRUE | FALSE | Uncharacterized protein | tr \| F6ZHQ5 \| | 27 kDa | TRUE | 14 |
| | | | OS = Equus caballus | F6ZHQ5_HORSE | | | |
| | | | GN = LOC100058853 PE = 4 SV = 1 | | | | |
| 94 | TRUE | FALSE | Uncharacterized protein | tr \| F7DZV9 \| | 28 kDa | TRUE | 13 |
| | | | OS = Equus caballus | F7DZV9_HORSE | | | |
| | | | GN = YWHAB PE = 3 SV = 1 | | | | |
| 95 | TRUE | FALSE | Uncharacterized protein | tr \| F7E3Y7 \| | 23 kDa | | 12 |
| | | | OS = Equus caballus | F7E3Y7_HORSE | | | |
| | | | GN = HSPB1 PE = 3 SV = 1 | | | | |
| 96 | TRUE | FALSE | Uncharacterized protein | tr \| F6U6L8 \| | 39 kDa | | 14 |
| | | | (Fragment) | F6U6L8_HORSE | | | |
| | | | OS = Equus caballus | | | | |
| | | | GN = CXADR PE = 4 SV = 1 | | | | |
| 97 | TRUE | FALSE | sp \| TRFL_HUMAN \| | sp \| TRFL_HUMAN \| | 78 kDa | TRUE | 7 |
| 98 | TRUE | FALSE | Uncharacterized protein | REVERSE_tr \| | 95 kDa | | 6 |
| | | | (Fragment) | F6RD43 \| | | | |
| | | | OS = Equus caballus | F6RD43_HORSE-R | | | |
| | | | GN = EIF2C4 PE = 4 SV = 1 | | | | |
| 99 | TRUE | FALSE | Adenylyl cyclase-associated | tr \| F6SRP7 \| | 51 kDa | | 13 |
| | | | protein | F6SRP7_HORSE | | | |
| | | | OS = Equus caballus | | | | |
| | | | GN = CAP1 PE = 3 SV = 1 | | | | |
| 100 | TRUE | FALSE | Uncharacterized protein | tr \| F6YAZ9 \| | 20 kDa | | 11 |
| | | | OS = Equus caballus | F6YAZ9_HORSE | | | |
| | | | GN = MYL12A PE = 4 SV = 1 | | | | |
| 101 | TRUE | FALSE | Serum albumin | sp \| P35747 \| | 69 kDa | TRUE | 8 |
| | | | OS = Equus caballus | ALBU_HORSE | | | |
| | | | GN = ALB PE = 1 SV = 1 | (+1) | | | |
| 102 | TRUE | FALSE | Annexin A1 | sp \| Q8HZM6 \| | 39 kDa | | 11 |
| | | | OS = Equus caballus | ANXA1_HORSE | | | |
| | | | GN = ANXA1 PE = 2 SV = 3 | | | | |
| 103 | TRUE | FALSE | Sodium/potassium-transporting | sp \| P18907 \| | 113 kDa | | 11 |
| | | | ATPase subunit alpha-1 | AT1A1_HORSE | | | |
| | | | OS = Equus caballus | (+1) | | | |
| | | | GN = ATP1A1 PE = 3 SV = 1 | | | | |
| 104 | TRUE | FALSE | 40S ribosomal protein S6 | tr \| F6V9Q3 \| | 29 kDa | | 12 |
| | | | (Fragment) | F6V9Q3_HORSE | | | |
| | | | OS = Equus caballus | | | | |
| | | | GN = RPS6 PE = 3 SV = 1 | | | | |
| 105 | TRUE | FALSE | Malate dehydrogenase | tr \| F7CZS6 \| | 36 kDa | | 11 |
| | | | OS = Equus caballus | F7CZS6_HORSE | | | |
| | | | GN = MDH1 PE = 3 SV = 1 | | | | |
| 106 | TRUE | FALSE | Uncharacterized protein | tr \| F6QAT0 \| | 341 kDa | | 9 |
| | | | OS = Equus caballus | F6QAT0_HORSE | | | |
| | | | GN = COL6A3 PE = 4 SV = 1 | (+1) | | | |
| 107 | TRUE | FALSE | Uncharacterized protein | tr \| F6YL06 \| | 37 kDa | | 12 |
| | | | OS = Equus caballus | F6YL06_HORSE | | | |
| | | | GN = GNB1 PE = 4 SV = 1 | | | | |
| 108 | TRUE | FALSE | 40S ribosomal protein S8 | tr \| F7DW15 \| | 22 kDa | | 12 |
| | | | (Fragment) | F7DW15_HORSE | | | |
| | | | OS = Equus caballus | | | | |
| | | | GN = RPS8 PE = 3 SV = 1 | | | | |
| 109 | TRUE | FALSE | Uncharacterized protein | tr \| F6WGL8 \| | 50 kDa | | 12 |
| | | | (Fragment) | F6WGL8_HORSE | | | |
| | | | OS = Equus caballus | | | | |
| | | | GN = PDIA4 PE = 3 SV = 1 | | | | |
| 110 | TRUE | FALSE | Uncharacterized protein | tr \| F6QDP0 \| | 122 kDa | | 12 |
| | | | (Fragment) | F6QDP0_HORSE | | | |
| | | | OS = Equus caballus | | | | |
| | | | GN = ACLY PE = 4 SV = 1 | | | | |
| 111 | TRUE | FALSE | Uncharacterized protein | tr \| F6XK12 \| | 53 kDa | | 9 |
| | | | OS = Equus caballus | F6XK12_HORSE | | | |
| | | | GN = SHMT2 PE = 3 SV = 1 | | | | |
| 112 | TRUE | FALSE | Uncharacterized protein | tr \| F6S3Q9 \| | 29 kDa | | 12 |
| | | | OS = Equus caballus | F6S3Q9_HORSE | | | |
| | | | GN = LOC100146711 PE = 3 SV = 1 | | | | |
| 113 | TRUE | FALSE | Uncharacterized protein | tr \| F6VVM5 \| | 156 kDa | | 11 |
| | | | (Fragment) | F6VVM5_HORSE | | | |
| | | | OS = Equus caballus | | | | |
| | | | GN = COL4A1 PE = 4 SV = 1 | | | | |
| 114 | TRUE | FALSE | Lumican | tr \| A2Q0Z2 \| | 39 kDa | | 11 |
| | | | OS = Equus caballus | A2Q0Z2_HORSE | | | |
| | | | GN = LUM PE = 2 SV = 1 | (+2) | | | |
| 115 | TRUE | FALSE | Uncharacterized protein | tr \| F6TCR6 \| | 47 kDa | | 11 |
| | | | OS = Equus caballus | F6TCR6_HORSE | | | |
| | | | GN = CKMT1B PE = 3 SV = 1 | | | | |
| 116 | TRUE | FALSE | Uncharacterized protein | tr \| F7CL80 \| | 18 kDa | TRUE | 9 |
| | | | (Fragment) | F7CL80_HORSE | | | |
| | | | OS = Equus caballus | | | | |
| | | | GN = DSTN PE = 4 SV = 1 | | | | |
| 117 | TRUE | FALSE | Uncharacterized protein | tr \| F7AVB5 \| | 19 kDa | | 9 |
| | | | OS = Equus caballus | F7AVB5_HORSE | | | |
| | | | GN = SRSF3 PE = 4 SV = 1 | | | | |
| 118 | TRUE | FALSE | Uncharacterized protein | tr \| F6Y2Q4 \| | 43 kDa | | 10 |
| | | | OS = Equus caballus | F6Y2Q4_HORSE | | | |
| | | | GN = CKB PE = 3 SV = 1 | | | | |
| 119 | TRUE | FALSE | Uncharacterized protein | tr \| F6XAY6 \| | 30 kDa | | 6 |
| | | | (Fragment) | F6XAY6_HORSE | | | |
| | | | OS = Equus caballus | | | | |
| | | | GN = SET PE = 3 SV = 1 | | | | |
| 120 | TRUE | FALSE | Uncharacterized protein | tr \| F6X6A6 \| | 11 kDa | TRUE | 9 |
| | | | OS = Equus caballus | F6X6A6_HORSE | | | |
| | | | GN = LOC100052020 PE = 3 SV = 1 | | | | |
| 121 | TRUE | FALSE | Uncharacterized protein | tr \| F7BFT1 \| | 22 kDa | | 11 |
| | | | OS = Equus caballus | F7BFT1_HORSE | | | |
| | | | GN = PRDX2 PE = 4 SV = 1 | | | | |
| 122 | TRUE | FALSE | Uncharacterized protein | tr \| F7DK89 \| | 19 kDa | | 6 |
| | | | (Fragment) | F7DK89_HORSE | | | |
| | | | OS = Equus caballus | | | | |
| | | | GN = TPT1 PE = 4 SV = 1 | | | | |
| 123 | TRUE | FALSE | Uncharacterized protein | tr \| F6Z587 \| | 61 kDa | | 9 |
| | | | OS = Equus caballus | F6Z587_HORSE | | | |
| | | | GN = HSPD1 PE = 3 SV = 1 | | | | |
| 124 | TRUE | FALSE | Uncharacterized protein | tr \| F6PN89 \| | 272 kDa | | 9 |
| | | | (Fragment) | F6PN89_HORSE | | | |
| | | | OS = Equus caballus | (+2) | | | |
| | | | GN = TLN1 PE = 4 SV = 1 | | | | |
| 125 | TRUE | FALSE | Uncharacterized protein | tr \| F6YHN4 \| | 28 kDa | | 10 |
| | | | OS = Equus caballus | F6YHN4_HORSE | | | |
| | | | GN = SRSF1 PE = 4 SV = 1 | | | | |
| 126 | TRUE | FALSE | Uncharacterized protein | tr \| F6YR34 \| | 130 kDa | | 7 |
| | | | OS = Equus caballus | F6YR34_HORSE | | | |
| | | | GN = THBS1 PE = 4 SV = 1 | | | | |
| 127 | TRUE | FALSE | Uncharacterized protein | tr \| F6YRC5 \| | 187 kDa | | 8 |
| | | | (Fragment) | F6YRC5_HORSE | | | |
| | | | OS = Equus caballus | | | | |
| | | | GN = IQGAP1 PE = 4 SV = 1 | | | | |
| 128 | TRUE | FALSE | Uncharacterized protein | tr \| F6W039 \| | 23 kDa | | 10 |
| | | | OS = Equus caballus | F6W039_HORSE | | | |
| | | | GN = ARHGDIA PE = 4 SV = 1 | | | | |
| 129 | TRUE | FALSE | Uncharacterized protein | tr \| F7C177 \| | 49 kDa | | 8 |
| | | | (Fragment) | F7C177_HORSE | | | |
| | | | OS = Equus caballus | | | | |
| | | | GN = SFPQ PE = 4 SV = 1 | | | | |
| 130 | TRUE | FALSE | Uncharacterized protein | tr \| F6UB53 \| | 20 kDa | | 4 |
| | | | (Fragment) | F6UB53_HORSE | | | |
| | | | OS = Equus caballus | | | | |
| | | | GN = RPL11 PE = 3 SV = 1 | | | | |
| 131 | TRUE | FALSE | Profilin | tr \| F6TEI8 \| | 15 kDa | | 9 |
| | | | OS = Equus caballus | F6TEI8_HORSE | | | |
| | | | GN = LOC100060955 PE = 3 | (+1) | | | |
| 132 | TRUE | FALSE | Uncharacterized protein | tr \| F6T114 \| | 49 kDa | TRUE | 6 |
| | | | OS = Equus caballus | F6T114_HORSE | | | |
| | | | GN = KRT20 PE = 3 SV = 1 | | | | |
| 133 | TRUE | FALSE | F-actin-capping protein | sp \| Q2QLA8 \| | 33 kDa | TRUE | 9 |
| | | | subunit alpha-2 | CAZA2_HORSE | | | |
| | | | OS = Equus caballus | | | | |
| | | | GN = CAPZA2 PE = 3 SV = 3 | | | | |
| 134 | TRUE | FALSE | Beta catenin 1 | tr \| B1MV73 \| | 86 kDa | | 8 |
| | | | OS = Equus caballus | B1MV73_HORSE | | | |
| | | | GN = CTNNB1 PE = 2 SV = 1 | | | | |
| 135 | TRUE | FALSE | ATP synthase subunit alpha | tr \| F6XUJ2 \| | 60 kDa | | 8 |
| | | | OS = Equus caballus | F6XUJ2_HORSE | | | |
| | | | GN = ATP5A1 PE = 3 SV = 1 | | | | |
| 136 | TRUE | FALSE | Uncharacterized protein | tr \| F6VS95 \| | 48 kDa | | 9 |
| | | | (Fragment) | F6VS95_HORSE | | | |
| | | | OS = Equus caballus | | | | |
| | | | GN = PDIA6 PE = 3 SV = 1 | | | | |
| 137 | TRUE | FALSE | Uncharacterized protein | tr \| F6VIG9 \| | 48 kDa | | 9 |
| | | | OS = Equus caballus | F6VIG9_HORSE | | | |
| | | | GN = LOC100054381 PE = 3 SV = 1 | (+1) | | | |
| 138 | TRUE | FALSE | Uncharacterized protein | tr \| F6Z8W0 \| | 66 kDa | | 8 |
| | | | (Fragment) | F6Z8W0_HORSE | | | |
| | | | OS = Equus caballus | | | | |
| | | | GN = WDR1 PE = 4 SV = 1 | | | | |
| 139 | TRUE | FALSE | Uncharacterized protein | tr \| F6SXD7 \| | 30 kDa | | 9 |
| | | | OS = Equus caballus | F6SXD7_HORSE | | | |
| | | | GN = PHB PE = 4 SV = 1 | | | | |
| 140 | TRUE | FALSE | Uncharacterized protein | tr \| F6TK05 \| | 58 kDa | | 9 |
| | | | (Fragment) | F6TK05_HORSE | | | |
| | | | OS = Equus caballus | | | | |
| | | | GN = CCT2 PE = 3 SV = 1 | | | | |
| 141 | TRUE | FALSE | Uncharacterized protein | tr \| F6PJF0 \| | 21 kDa | | 9 |
| | | | OS = Equus caballus | F6PJF0_HORSE | | | |
| | | | GN = CBX3 PE = 4 SV = 1 | | | | |
| 142 | TRUE | FALSE | Uncharacterized protein | tr \| F6XXH8 \| | 33 kDa | | 7 |
| | | | OS = Equus caballus | F6XXH8_HORSE | | | |
| | | | GN = PHB2 PE = 4 SV = 1 | | | | |
| 143 | TRUE | FALSE | Uncharacterized protein | tr \| F6S9X8 \| | 16 kDa | | 9 |
| | | | OS = Equus caballus | F6S9X8_HORSE | | | |
| | | | GN = COTL1 PE = 4 SV = 1 | | | | |
| 144 | TRUE | FALSE | Uncharacterized protein | tr \| F7CC59 \| | 52 kDa | | 4 |
| | | | OS = Equus caballus | F7CC59_HORSE | | | |
| | | | GN = TINAGL1 PE = 3 SV = 1 | | | | |
| 145 | TRUE | FALSE | Uncharacterized protein | tr \| F7CBN0 \| | 37 kDa | | 7 |
| | | | OS = Equus caballus | F7CBN0_HORSE | | | |
| | | | GN = AKR1A1 PE = 4 SV = 1 | | | | |
| 146 | TRUE | FALSE | Uncharacterized protein | tr \| F7AHF3 \| | 42 kDa | TRUE | 4 |
| | | | OS = Equus caballus | F7AHF3_HORSE | | | |
| | | | GN = ACTBL2 PE = 3 SV = 1 | | | | |
| 147 | TRUE | FALSE | Uncharacterized protein | tr \| F6XH19 \| | 135 kDa | | 7 |
| | | | OS = Equus caballus | F6XH19_HORSE | | | |
| | | | GN = NID1 PE = 4 SV = 1 | | | | |
| 148 | TRUE | FALSE | Uncharacterized protein | tr \| F7BXV9 \| | 387 kDa | | 8 |
| | | | (Fragment) | F7BXV9_HORSE | | | |
| | | | OS = Equus caballus | | | | |
| | | | GN = LAMA5 PE = 4 SV = 1 | | | | |
| 149 | TRUE | FALSE | Uncharacterized protein | tr \| F6Z4J4 \| | 49 kDa | | 7 |
| | | | (Fragment) | F6Z4J4_HORSE | | | |
| | | | OS = Equus caballus | | | | |
| | | | GN = GDI2 PE = 4 SV = 1 | | | | |
| 150 | TRUE | FALSE | Aspartate aminotransferase, | sp \| P08907 \| | 45 kDa | | 7 |
| | | | mitochondrial | AATM_HORSE | | | |
| | | | OS = Equus caballus | (+1) | | | |
| | | | GN = GOT2 PE = 1 SV = 1 | | | | |
| 151 | TRUE | FALSE | Uncharacterized protein | tr \| F7BCM8 \| | 46 kDa | | 6 |
| | | | OS = Equus caballus | F7BCM8_HORSE | | | |
| | | | GN = OLFML3 PE = 4 SV = 1 | | | | |
| 152 | TRUE | FALSE | Uncharacterized protein | tr \| F6X9D3 \| | 33 kDa | | 6 |
| | | | OS = Equus caballus | F6X9D3_HORSE | | | |
| | | | GN = NPM1 PE = 4 SV = 1 | | | | |
| 153 | TRUE | FALSE | Uncharacterized protein | tr \| F6XAL9 \| | 70 kDa | | 8 |
| | | | (Fragment) | F6XAL9_HORSE | | | |
| | | | OS = Equus caballus | (+1) | | | |
| | | | GN = HNRNPM PE = 4 SV = 1 | | | | |
| 154 | TRUE | FALSE | Uncharacterized protein | tr \| F6ZQE2 \| | 60 kDa | | 8 |
| | | | OS = Equus caballus | F6ZQE2_HORSE | | | |
| | | | GN = CCT8 PE = 3 SV = 1 | | | | |
| 155 | TRUE | FALSE | Uncharacterized protein | tr \| F6Y739 \| | 31 kDa | | 6 |
| | | | OS = Equus caballus | F6Y739_HORSE | | | |
| | | | GN = RPS2 PE = 3 SV = 1 | | | | |
| 156 | TRUE | FALSE | Fascin (Fragment) | tr \| F6TLB5 \| | 55 kDa | | 7 |
| | | | OS = Equus caballus | F6TLB5_HORSE | | | |
| | | | GN = FSCN1 PE = 3 SV = 1 | | | | |
| 157 | TRUE | FALSE | Hemoglobin subunit alpha-2 | sp \| Q9TVA3 \| | 15 kDa | TRUE | 1 |
| | | | OS = Equus burchelli | HBA2_EQUBU | | | |
| | | | GN = HBA2 PE = 2 | | | | |
| 158 | TRUE | FALSE | Uncharacterized protein | tr \| F6VG02 \| | 3912 kDa | | 1 |
| | | | OS = Equus caballus | F6VG02_HORSE | | | |
| | | | PE = 4 SV = 1 | | | | |
| 159 | TRUE | FALSE | Uncharacterized protein | tr \| F6YQC6 \| | 100 kDa | | 8 |
| | | | OS = Equus caballus | F6YQC6_HORSE | | | |
| | | | GN = CTNNA1 PE = 4 SV = 1 | | | | |
| 160 | TRUE | FALSE | Uncharacterized protein | tr \| F7CB51 \| | 30 kDa | | 8 |
| | | | OS = Equus caballus | F7CB51_HORSE | | | |
| | | | GN = BPGM PE = 3 SV = 1 | | | | |
| 161 | TRUE | FALSE | Uncharacterized protein | tr \| F6QL56 \| | 34 kDa | | 6 |
| | | | OS = Equus caballus | F6QL56_HORSE | | | |
| | | | GN = TRA2B PE = 4 SV = 1 | | | | |
| 162 | TRUE | FALSE | Histone H2B | tr \| F6VYH8 \| | 14 kDa | TRUE | 8 |
| | | | OS = Equus caballus | F6VYH8_HORSE | | | |
| | | | GN = HIST2H2BF PE = 3 SV = 1 | | | | |
| 163 | TRUE | FALSE | Uncharacterized protein | tr \| F6WAT9 \| | 50 kDa | | 1 |
| | | | OS = Equus caballus | F6WAT9_HORSE | | | |
| | | | GN = PPARD PE = 3 SV = 1 | | | | |
| 164 | TRUE | FALSE | Uncharacterized protein | tr \| F6VWK4 \| | 37 kDa | | 7 |
| | | | (Fragment) | F6VWK4_HORSE | | | |
| | | | OS = Equus caballus | | | | |
| | | | GN = HNRNPA3 PE = 4 SV = 1 | | | | |
| 165 | TRUE | FALSE | Fibronectin | tr \| F7CN05 \| | 253 kDa | | 6 |
| | | | OS = Equus caballus | F7CN05_HORSE | | | |
| | | | GN = FN1 PE = 4 SV = 1 | (+2) | | | |
| 166 | TRUE | FALSE | Glutamine synthetase | tr \| F6TAZ0 \| | 43 kDa | | 6 |
| | | | (Fragment) | F6TAZ0_HORSE | | | |
| | | | OS = Equus caballus | | | | |
| | | | GN = GLUL PE = 3 SV = 1 | | | | |
| 167 | TRUE | FALSE | sp \| ANXA5_HUMAN \| | sp \| ANXA5_HUMAN \| | 36 kDa | | 5 |
| 168 | TRUE | FALSE | Uncharacterized protein | tr \| F6TFH2 \| | 76 kDa | | 4 |
| | | | (Fragment) | F6TFH2_HORSE | | | |
| | | | OS = Equus caballus | | | | |
| | | | GN = CPXM1 PE = 4 SV = 1 | | | | |
| 169 | TRUE | FALSE | Uncharacterized protein | tr \| F7C6F2 \| | 118 kDa | | 4 |
| | | | OS = Equus caballus | F7C6F2_HORSE | | | |
| | | | GN = UBA1 PE = 3 SV = 1 | | | | |
| 170 | TRUE | FALSE | Uncharacterized protein | tr \| F6VYB1 \| | 37 kDa | | 3 |
| | | | (Fragment) | F6VYB1_HORSE | | | |
| | | | OS = Equus caballus | | | | |
| | | | GN = HNRNPA2B1 PE = 4 SV = 1 | | | | |
| 171 | TRUE | FALSE | Uncharacterized protein | tr \| F6RA08 \| | 31 kDa | | 7 |
| | | | OS = Equus caballus | F6RA08_HORSE | | | |
| | | | GN = STOM PE = 4 SV = 1 | | | | |
| 172 | TRUE | FALSE | Uncharacterized protein | tr \| F6SSK7 \| | 21 kDa | | 6 |
| | | | OS = Equus caballus | F6SSK7_HORSE | | | |
| | | | GN = RAP1B PE = 4 SV = 1 | | | | |
| 173 | TRUE | FALSE | Uncharacterized protein | tr \| F6TSK0 \| | 17 kDa | | 4 |
| | | | OS = Equus caballus | F6TSK0_HORSE | | | |
| | | | GN = RPL26 PE = 3 SV = 1 | | | | |
| 174 | TRUE | FALSE | Uncharacterized protein | tr \| F6Z1Z7 \| | 30 kDa | | 7 |
| | | | OS = Equus caballus | F6Z1Z7_HORSE | | | |
| | | | GN = RPS3A PE = 3 SV = 1 | | | | |
| 175 | TRUE | FALSE | Uncharacterized protein | tr \| F7B9P4 \| | 24 kDa | TRUE | 7 |
| | | | (Fragment) | F7B9P4_HORSE | | | |
| | | | OS = Equus caballus | | | | |
| | | | GN = RAB14 PE = 3 SV = 1 | | | | |
| 176 | TRUE | FALSE | Uncharacterized protein | tr \| F6Q698 \| | 99 kDa | | 2 |
| | | | OS = Equus caballus | F6Q698_HORSE | | | |
| | | | GN = MVP PE = 4 SV = 1 | | | | |
| 177 | TRUE | FALSE | Coronin | tr \| F7CVF5 \| | 54 kDa | TRUE | 6 |
| | | | OS = Equus caballus | F7CVF5_HORSE | | | |
| | | | GN = CORO1B PE = 3 SV = 1 | | | | |
| 178 | TRUE | FALSE | Uncharacterized protein | tr \| F6RMN2 \| | 49 kDa | | 6 |
| | | | OS = Equus caballus | F6RMN2_HORSE | | | |
| | | | GN = DBNL PE = 4 SV = 1 | | | | |
| 179 | TRUE | FALSE | Transgelin | tr \| A8J714 \| | 23 kDa | | 7 |
| | | | OS = Equus caballus | A8J714_HORSE | | | |
| | | | GN = TAGLN PE = 2 SV = 1 | | | | |
| 180 | TRUE | FALSE | Uncharacterized protein | tr \| F6S535 \| | 25 kDa | | 7 |
| | | | OS = Equus caballus | F6S535_HORSE | | | |
| | | | GN = TMED10 PE = 3 SV = 1 | | | | |
| 181 | TRUE | FALSE | Histone H2A | tr \| F6SWA7 \| | 14 kDa | TRUE | 7 |
| | | | OS = Equus caballus | F6SWA7_HORSE | | | |
| | | | GN = LOC100053206 PE = 3 SV = 1 | (+3) | | | |
| 182 | TRUE | FALSE | Uncharacterized protein | tr \| F6T1A4 \| | 18 kDa | | 7 |
| | | | OS = Equus caballus | F6T1A4_HORSE | | | |
| | | | GN = LOC100070538 PE = 3 SV = 1 | | | | |
| 183 | TRUE | FALSE | Uncharacterized protein | tr \| F6XWC6 \| | 29 kDa | | 7 |
| | | | (Fragment) | F6XWC6_HORSE | | | |
| | | | OS = Equus caballus | | | | |
| | | | GN = SRSF6 PE = 4 SV = 1 | | | | |
| 184 | TRUE | FALSE | Uncharacterized protein | tr \| F6YY66 \| | 26 kDa | | 5 |
| | | | OS = Equus caballus | F6YY66_HORSE | | | |
| | | | GN = NME2 PE = 3 SV = 1 | | | | |
| 185 | TRUE | FALSE | Uncharacterized protein | tr \| F7CMH2 \| | 44 kDa | | 7 |
| | | | OS = Equus caballus | F7CMH2_HORSE | | | |
| | | | GN = PA2G4 PE = 4 SV = 1 | | | | |
| 186 | TRUE | FALSE | 6-phosphogluconate dehydrogenase, | tr \| F7D917 \| | 53 kDa | | 7 |
| | | | decarboxylating (Fragment) | F7D917_HORSE | | | |
| | | | OS = Equus caballus | | | | |
| | | | GN = PGD PE = 3 SV = 1 | | | | |
| 187 | TRUE | FALSE | Decorin | sp \| O46542 \| | 40 kDa | | 4 |
| | | | OS = Equus caballus | PGS2_HORSE | | | |
| | | | GN = DCN PE = 2 SV = 1 | | | | |
| 188 | TRUE | FALSE | Uncharacterized protein | tr \| F6WQX2 \| | 842 kDa | | 6 |
| | | | OS = Equus caballus | F6WQX2_HORSE | | | |
| | | | GN = OBSCN PE = 4 SV = 1 | | | | |
| 189 | TRUE | FALSE | Uncharacterized protein | tr \| F6Q448 \| | 8 kDa | | 6 |
| | | | (Fragment) | F6Q448_HORSE | | | |
| | | | OS = Equus caballus | | | | |
| | | | PE = 4 SV = 1 | | | | |
| 190 | TRUE | FALSE | Uncharacterized protein | tr \| F6UPM7 \| | 284 kDa | | 6 |
| | | | OS = Equus caballus | F6UPM7_HORSE | | | |
| | | | GN = SPTAN1 PE = 4 SV = 1 | (+3) | | | |
| 191 | TRUE | FALSE | Uncharacterized protein | tr \| F6ZXJ2 \| | 305 kDa | TRUE | 6 |
| | | | (Fragment) | F6ZXJ2_HORSE | | | |
| | | | OS = Equus caballus | (+4) | | | |
| | | | GN = FBN2 PE = 4 SV = 1 | | | | |
| 192 | TRUE | FALSE | Integrin beta (Fragment) | tr \| F6UR46 \| | 88 kDa | | 4 |
| | | | OS = Equus caballus | F6UR46_HORSE | | | |
| | | | GN = ITGB1 PE = 3 SV = 1 | | | | |
| 193 | TRUE | FALSE | Uncharacterized protein | tr \| F6QHQ9 \| | 250 kDa | | 4 |
| | | | OS = Equus caballus | F6QHQ9_HORSE | | | |
| | | | GN = SPTBN1 PE = 4 SV = 1 | (+6) | | | |
| 194 | TRUE | FALSE | Uncharacterized protein | tr \| F6UCB3 \| | 27 kDa | | 4 |
| | | | (Fragment) | F6UCB3_HORSE | | | |
| | | | OS = Equus caballus | (+1) | | | |
| | | | GN = RPL7A PE = 4 SV = 1 | | | | |
| 195 | TRUE | FALSE | sp \| CAH2_BOVIN \| | sp \| CAH2_BOVIN \| | 29 kDa | | 6 |
| 196 | TRUE | FALSE | Uncharacterized protein | tr \| F6S3E5 \| | 71 kDa | TRUE | 4 |
| | | | OS = Equus caballus | F6S3E5_HORSE | | | |
| | | | GN = RDX PE = 4 SV = 1 | | | | |
| 197 | TRUE | FALSE | Uncharacterized protein | tr \| F6S8T9 \| | 38 kDa | TRUE | 6 |
| | | | (Fragment) | F6S8T9_HORSE | | | |
| | | | OS = Equus caballus | | | | |
| | | | GN = ERLIN2 PE = 4 SV = 1 | | | | |
| 198 | TRUE | FALSE | Uncharacterized protein | tr \| F6X7C1 \| | 32 kDa | | 6 |
| | | | (Fragment) | F6X7C1_HORSE | | | |
| | | | OS = Equus caballus | | | | |
| | | | GN = RCN2 PE = 4 SV = 1 | | | | |
| 199 | TRUE | FALSE | Uncharacterized protein | tr \| F6Y4J0 \| | 52 kDa | | 6 |
| | | | (Fragment) | F6Y4J0_HORSE | | | |
| | | | OS = Equus caballus | | | | |
| | | | GN = ATP6V1B2 PE = 3 SV = 1 | | | | |
| 200 | TRUE | FALSE | Uncharacterized protein | tr \| F7DQS6 \| | 24 kDa | | 6 |
| | | | (Fragment) | F7DQS6_HORSE | | | |
| | | | OS = Equus caballus | | | | |
| | | | GN = LOC100070738 PE = 3 SV = 1 | | | | |
| 201 | TRUE | FALSE | Uncharacterized protein | tr \| F6UUX5 \| | 46 kDa | | 4 |
| | | | (Fragment) | F6UUX5_HORSE | | | |
| | | | OS = Equus caballus | | | | |
| | | | GN = RPL3 PE = 3 SV = 1 | | | | |
| 202 | TRUE | FALSE | Uncharacterized protein | tr \| F6WB41 \| | 27 kDa | | 6 |
| | | | OS = Equus caballus | F6WB41_HORSE | | | |
| | | | GN = RPL8 PE = 4 SV = 1 | | | | |
| 203 | TRUE | FALSE | Uncharacterized protein | tr \| F7CG77 \| | 58 kDa | | 5 |
| | | | OS = Equus caballus | F7CG77_HORSE | | | |
| | | | GN = CCT6A PE = 3 SV = 1 | | | | |
| 204 | TRUE | FALSE | Cathepsin B (Fragment) | tr \| B7X6D1 \| | 18 kDa | | 5 |
| | | | OS = Equus caballus | B7X6D1_HORSE | | | |
| | | | GN = CTSB PE = 2 SV = 1 | (+1) | | | |
| 205 | TRUE | FALSE | Uncharacterized protein | tr \| F6SHH7 \| | 42 kDa | | 6 |
| | | | OS = Equus caballus | F6SHH7_HORSE | | | |
| | | | GN = SEPT2 PE = 3 SV = 1 | | | | |
| 206 | TRUE | FALSE | Uncharacterized protein | tr \| F6V3D5 \| | 61 kDa | | 6 |
| | | | OS = Equus caballus | F6V3D5_HORSE | | | |
| | | | GN = CCT3 PE = 3 SV = 1 | | | | |
| 207 | TRUE | FALSE | Uncharacterized protein | tr \| F6VT29 \| | 51 kDa | TRUE | 4 |
| | | | (Fragment) | F6VT29_HORSE | | | |
| | | | OS = Equus caballus | | | | |
| | | | GN = LOC100061458 PE = 3 SV = 1 | | | | |
| 208 | TRUE | FALSE | Uncharacterized protein | tr \| F6X756 \| | 17 kDa | | 5 |
| | | | (Fragment) | F6X756_HORSE | | | |
| | | | OS = Equus caballus | | | | |
| | | | GN = EEF1B2 PE = 3 SV = 1 | | | | |
| 209 | TRUE | FALSE | Ribosomal protein L15 | tr \| F6RMA4 \| | 24 kDa | | 5 |
| | | | OS = Equus caballus | F6RMA4_HORSE | | | |
| | | | GN = LOC100051688 PE = 3 SV = 1 | (+1) | | | |
| 210 | TRUE | FALSE | Histone H2B | tr \| F6PWV1 \| | 14 kDa | TRUE | 5 |
| | | | OS = Equus caballus | F6PWV1_HORSE | | | |
| | | | GN = LOC100052889 PE = 3 SV = 1 | | | | |
| 211 | TRUE | FALSE | Uncharacterized protein | tr \| F6QHY8 \| | 26 kDa | TRUE | 6 |
| | | | (Fragment) | F6QHY8_HORSE | | | |
| | | | OS = Equus caballus | | | | |
| | | | GN = CLIC4 PE = 4 SV = 1 | | | | |
| 212 | TRUE | FALSE | Histone H2B | tr \| F6VUX0 \| | 14 kDa | TRUE | 6 |
| | | | OS = Equus caballus | F6VUX0_HORSE | | | |
| | | | GN = LOC100068222 PE = 3 SV = 1 | (+1) | | | |
| 213 | TRUE | FALSE | Uncharacterized protein | tr \| F6WY40 \| | 47 kDa | | 6 |
| | | | OS = Equus caballus | F6WY40_HORSE | | | |
| | | | GN = ERP44 PE = 4 SV = 1 | (+1) | | | |
| 214 | TRUE | FALSE | Uncharacterized protein | tr \| F7CW82 \| | 42 kDa | TRUE | 6 |
| | | | OS = Equus caballus | F7CW82_HORSE | | | |
| | | | GN = ACTA2 PE = 3 SV = 1 | | | | |
| 215 | TRUE | FALSE | Histone H2B | tr \| F7E1X9 \| | 14 kDa | TRUE | 6 |
| | | | OS = Equus caballus | F7E1X9_HORSE | | | |
| | | | GN = HIST1H2BD PE = 3 SV = 1 | | | | |
| 216 | TRUE | FALSE | Peptidyl-prolyl cis-trans | tr \| A5YBL8 \| | 24 kDa | | 5 |
| | | | isomerase | A5YBL8_HORSE | | | |
| | | | OS = Equus caballus | | | | |
| | | | GN = PPIB PE = 1 SV = 1 | | | | |
| 217 | TRUE | FALSE | Uncharacterized protein | tr \| F6TTX8 \| | 12 kDa | | 5 |
| | | | OS = Equus caballus | F6TTX8_HORSE | | | |
| | | | GN = LOC100146851 PE = 3 SV = 1 | | | | |
| 218 | TRUE | FALSE | Uncharacterized protein | tr \| F6V8I5 \| | 37 kDa | | 5 |
| | | | OS = Equus caballus | F6V8I5_HORSE | | | |
| | | | GN = PCBP1 PE = 4 SV = 1 | | | | |
| 219 | TRUE | FALSE | Uncharacterized protein | tr \| F7A676 \| | 56 kDa | | 5 |
| | | | OS = Equus caballus | F7A676_HORSE | | | |
| | | | GN = LAP3 PE = 3 SV = 1 | | | | |
| 220 | TRUE | FALSE | Uncharacterized protein | tr \| F6PQD3 \| | 16 kDa | | 4 |
| | | | (Fragment) | F6PQD3_HORSE | | | |
| | | | OS = Equus caballus | (+1) | | | |
| | | | GN = LOC100068442 PE = 3 SV = 1 | | | | |
| 221 | TRUE | FALSE | Uncharacterized protein | tr \| F6TLU0 \| | 32 kDa | | 5 |
| | | | (Fragment) | F6TLU0_HORSE | | | |
| | | | OS = Equus caballus | | | | |
| | | | GN = VDAC2 PE = 4 SV = 1 | | | | |
| 222 | TRUE | FALSE | sp \| PRDX1_HUMAN \| | sp \| PRDX1_HUMAN \| | 22 kDa | TRUE | 5 |
| | | | | (+1) | | | |
| 223 | TRUE | FALSE | Uncharacterized protein | tr \| F6UH83 \| | 60 kDa | | 5 |
| | | | OS = Equus caballus | F6UH83_HORSE | | | |
| | | | GN = CCT5 PE = 3 SV = 1 | | | | |
| 224 | TRUE | FALSE | Fibrinopeptide B | tr \| F6PH38 \| | 56 kDa | | 4 |
| | | | (Fragment) | F6PH38_HORSE | | | |
| | | | OS = Equus caballus | | | | |
| | | | GN = FGB PE = 4 SV = 1 | | | | |
| 225 | TRUE | FALSE | Uncharacterized protein | tr \| F6WPT4 \| | 22 kDa | | 2 |
| | | | OS = Equus caballus | F6WPT4_HORSE | | | |
| | | | GN = PGRMC1 PE = 4 SV = 1 | | | | |
| 226 | TRUE | FALSE | Uncharacterized protein | tr \| F6ZY52 \| | 51 kDa | | 1 |
| | | | (Fragment) | F6ZY52_HORSE | | | |
| | | | OS = Equus caballus | | | | |
| | | | GN = SUCLA2 PE = 3 SV = 1 | | | | |
| 227 | TRUE | FALSE | Uncharacterized protein | tr \| F7BTK9 \| | 84 kDa | | 2 |
| | | | OS = Equus caballus | F7BTK9_HORSE | | | |
| | | | GN = FAM129A PE = 4 SV = 1 | (+1) | | | |
| 228 | TRUE | FALSE | Uncharacterized protein | tr \| F7C1V1 \| | 104 kDa | | 1 |
| | | | (Fragment) | F7C1V1_HORSE | | | |
| | | | OS = Equus caballus | | | | |
| | | | GN = PRPF6 PE = 4 SV = 1 | | | | |
| 229 | TRUE | FALSE | Uncharacterized protein | tr \| F6YUH7 \| | 116 kDa | | 4 |
| | | | (Fragment) | F6YUH7_HORSE | | | |
| | | | OS = Equus caballus | | | | |
| | | | GN = HIP1R PE = 4 SV = 1 | | | | |
| 230 | TRUE | FALSE | Uncharacterized protein | tr \| F7B4L9 \| | 27 kDa | | 5 |
| | | | OS = Equus caballus | F7B4L9_HORSE | | | |
| | | | GN = LOC100147454 PE = 3 SV = 1 | (+1) | | | |
| 231 | TRUE | FALSE | Uncharacterized protein | tr \| F7D301 \| | 20 kDa | | 5 |
| | | | OS = Equus caballus | F7D301_HORSE | | | |
| | | | GN = RAB11B PE = 3 SV = 1 | | | | |
| 232 | TRUE | FALSE | Uncharacterized protein | tr \| F7DU34 \| | 18 kDa | | 5 |
| | | | OS = Equus caballus | F7DU34_HORSE | | | |
| | | | GN = RPS11 PE = 3 SV = 1 | | | | |
| 233 | TRUE | FALSE | Angiomodulin (Fragment) | tr \| Q95LJ1 \| | 13 kDa | | 5 |
| | | | OS = Equus caballus | Q95LJ1_HORSE | | | |
| | | | GN = AGM PE = 2 SV = 1 | | | | |
| 234 | TRUE | FALSE | Uncharacterized protein | tr \| F6QDX4 \| | 22 kDa | | 3 |
| | | | OS = Equus caballus | F6QDX4_HORSE | | | |
| | | | GN = TMED7 PE = 3 SV = 1 | | | | |
| 235 | TRUE | FALSE | Ribosomal protein (Fragment) | tr \| F7BFM4 \| | 25 kDa | | 4 |
| | | | OS = Equus caballus | F7BFM4_HORSE | | | |
| | | | GN = RPL10A PE = 3 SV = 1 | | | | |
| 236 | TRUE | FALSE | Uncharacterized protein | tr \| F6WTL6 \| | 60 kDa | | 2 |
| | | | OS = Equus caballus | F6WTL6_HORSE | | | |
| | | | GN = PRKCSH PE = 4 SV = 1 | | | | |
| 237 | TRUE | FALSE | Uncharacterized protein | REVERSE_tr \| | 57 kDa | | 3 |
| | | | OS = Equus caballus | F6TN81 \| | | | |
| | | | GN = LIPG PE = 3 SV = 1 | F6TN81_HORSE-R | | | |
| 238 | TRUE | FALSE | Uncharacterized protein | tr \| F6RUP0 \| | 27 kDa | TRUE | 5 |
| | | | (Fragment) | F6RUP0_HORSE | | | |
| | | | OS = Equus caballus | | | | |
| | | | GN = PRDX4 PE = 4 SV = 1 | | | | |
| 239 | TRUE | FALSE | Uncharacterized protein | tr \| F6S4R4 \| | 19 kDa | | 4 |
| | | | OS = Equus caballus | F6S4R4_HORSE | | | |
| | | | GN = SKP1 PE = 4 SV = 1 | | | | |
| 240 | TRUE | FALSE | Uncharacterized protein | tr \| F6VQF0 \| | 23 kDa | TRUE | 5 |
| | | | OS = Equus caballus | F6VQF0_HORSE | | | |
| | | | GN = RAB10 PE = 3 SV = 1 | | | | |
| 241 | TRUE | FALSE | Uncharacterized protein | tr \| F6VUR4 \| | 51 kDa | | 5 |
| | | | (Fragment) | F6VUR4_HORSE | | | |
| | | | OS = Equus caballus | | | | |
| | | | GN = CKAP4 PE = 4 SV = 1 | | | | |
| 242 | TRUE | FALSE | Uncharacterized protein | tr \| F6W7V0 \| | 62 kDa | TRUE | 5 |
| | | | OS = Equus caballus | F6W7V0_HORSE | | | |
| | | | GN = KRT5 PE = 3 SV = 1 | | | | |
| 243 | TRUE | FALSE | Uncharacterized protein | tr \| F7CS30 \| | 43 kDa | | 5 |
| | | | OS = Equus caballus | F7CS30_HORSE | | | |
| | | | GN = PTRF PE = 4 SV = 1 | | | | |
| 244 | TRUE | FALSE | 78 kDa glucose-regulated protein | tr \| Q9N1V8 \| | 11 kDa | TRUE | 4 |
| | | | (Fragment) | Q9N1V8_HORSE | | | |
| | | | OS = Equus caballus | | | | |
| | | | GN = GRP78 PE = 3 SV = 1 | | | | |
| 245 | TRUE | FALSE | Uncharacterized protein | tr \| F7BK38 \| | 196 kDa | | 4 |
| | | | OS = Equus caballus | F7BK38_HORSE | | | |
| | | | GN = LAMB2 PE = 4 SV = 1 | | | | |
| 246 | TRUE | FALSE | Uncharacterized protein | tr \| F6Q4L3 \| | 71 kDa | | 4 |
| | | | (Fragment) | F6Q4L3_HORSE | | | |
| | | | OS = Equus caballus | | | | |
| | | | GN = DDX17 PE = 3 SV = 1 | | | | |
| 247 | TRUE | FALSE | Uncharacterized protein | tr \| F6X3K0 \| | 69 kDa | | 4 |
| | | | (Fragment) | F6X3K0_HORSE | | | |
| | | | OS = Equus caballus | | | | |
| | | | GN = NCL PE = 4 SV = 1 | | | | |
| 248 | TRUE | FALSE | Uncharacterized protein | tr \| F6RHB3 \| | 532 kDa | | 4 |
| | | | OS = Equus caballus | F6RHB3_HORSE | | | |
| | | | GN = DYNC1H1 PE = 4 SV = 1 | (+1) | | | |
| 249 | TRUE | FALSE | Ribosomal protein L19 | tr \| F6QDH0 \| | 23 kDa | | 3 |
| | | | (Fragment) | F6QDH0_HORSE | | | |
| | | | OS = Equus caballus | (+1) | | | |
| | | | GN = LOC100630388 PE = 3 SV = 1 | | | | |
| 250 | TRUE | FALSE | Uncharacterized protein | tr \| H9H036 \| | 16 kDa | | 4 |
| | | | OS = Equus caballus | H9H036_HORSE | | | |
| | | | GN = RPL28 PE = 4 SV = 1 | | | | |
| 251 | TRUE | FALSE | Uncharacterized protein | tr \| F6W9B1 \| | 41 kDa | | 3 |
| | | | OS = Equus caballus | F6W9B1_HORSE | | | |
| | | | GN = ST13 PE = 4 SV = 1 | | | | |
| 252 | TRUE | FALSE | Uncharacterized protein | tr \| F7BA08 \| | 71 kDa | | 3 |
| | | | OS = Equus caballus | F7BA08_HORSE | | | |
| | | | GN = HNRNPR PE = 4 SV = 1 | | | | |
| 253 | TRUE | FALSE | Dihydrolipoyl dehydrogenase | tr \| F6U3F1 \| | 54 kDa | | 1 |
| | | | OS = Equus caballus | F6U3F1_HORSE | | | |
| | | | GN = DLD PE = 3 SV = 1 | | | | |
| 254 | TRUE | FALSE | Thymosin beta-4 | sp \| P62327 \| | 5 kDa | | 4 |
| | | | OS = Equus caballus | TYB4_HORSE | | | |
| | | | GN = TMSB4 PE = 1 SV = 2 | | | | |
| 255 | TRUE | FALSE | Uncharacterized protein | tr \| F6SEM5 \| | 162 kDa | | 4 |
| | | | (Fragment) | F6SEM5_HORSE | | | |
| | | | OS = Equus caballus | | | | |
| | | | GN = MRC2 PE = 4 SV = 1 | | | | |
| 256 | TRUE | FALSE | Uncharacterized protein | tr \| F6TNE1 \| | 23 kDa | | 4 |
| | | | OS = Equus caballus | F6TNE1_HORSE | | | |
| | | | GN = RPS5 PE = 3 SV = 1 | | | | |
| 257 | TRUE | FALSE | Uncharacterized protein | tr \| F6U3T9 \| | 35 kDa | | 4 |
| | | | (Fragment) | F6U3T9_HORSE | | | |
| | | | OS = Equus caballus | | | | |
| | | | GN = RPL5 PE = 3 SV = 1 | | | | |
| 258 | TRUE | FALSE | Uncharacterized protein | tr \| F6WCY9 \| | 49 kDa | | 2 |
| | | | OS = Equus caballus | F6WCY9_HORSE | | | |
| | | | GN = RNH1 PE = 4 SV = 1 | | | | |
| 259 | TRUE | FALSE | Uncharacterized protein | tr \| F6X9U4 \| | 97 kDa | | 3 |
| | | | OS = Equus caballus | F6X9U4_HORSE | | | |
| | | | GN = DAG1 PE = 4 SV = 1 | | | | |
| 260 | TRUE | FALSE | Uncharacterized protein | tr \| F6XLM5 \| | 16 kDa | | 4 |
| | | | OS = Equus caballus | F6XLM5_HORSE | | | |
| | | | GN = RPS14 PE = 3 SV = 1 | | | | |
| 261 | TRUE | FALSE | Citrate synthase | tr \| F6XMS1 \| | 52 kDa | | 4 |
| | | | OS = Equus caballus | F6XMS1_HORSE | | | |
| 262 | TRUE | FALSE | Uncharacterized protein | tr \| F6YAZ0 \| | 19 kDa | | 3 |
| | | | OS = Equus caballus | F6YAZ0_HORSE | | | |
| | | | GN = ATP5H PE = 4 SV = 1 | | | | |
| 263 | TRUE | FALSE | Uncharacterized protein | tr \| F6ZFH9 \| | 25 kDa | TRUE | 4 |
| | | | (Fragment) | F6ZFH9_HORSE | | | |
| | | | OS = Equus caballus | | | | |
| | | | GN = YWHAG PE = 3 SV = 1 | | | | |
| 264 | TRUE | FALSE | Uncharacterized protein | tr \| F6ZN26 \| | 23 kDa | | 4 |
| | | | (Fragment) | F6ZN26_HORSE | | | |
| | | | OS = Equus caballus | | | | |
| | | | GN = HDGF PE = 4 SV = 1 | | | | |
| 265 | TRUE | FALSE | Uncharacterized protein | tr \| F7AGA5 \| | 71 kDa | TRUE | 4 |
| | | | OS = Equus caballus | F7AGA5_HORSE | | | |
| | | | GN = PLS3 PE = 4 SV = 1 | | | | |
| 266 | TRUE | FALSE | Uncharacterized protein | tr \| F7DA17 \| | 78 kDa | | 4 |
| | | | OS = Equus caballus | F7DA17_HORSE | | | |
| | | | GN = TGM2 PE = 3 SV = 1 | | | | |
| 267 | TRUE | FALSE | Uncharacterized protein | tr \| F7DX98 \| | 43 kDa | | 4 |
| | | | OS = Equus caballus | F7DX98_HORSE | | | |
| | | | GN = PRKAR1A PE = 4 SV = 1 | | | | |
| 268 | TRUE | FALSE | Uncharacterized protein | tr \| F6Y2H3 \| | 55 kDa | | 3 |
| | | | (Fragment) | F6Y2H3_HORSE | | | |
| | | | OS = Equus caballus | (+1) | | | |
| | | | GN = PEPD PE = 3 SV = 1 | | | | |
| 269 | TRUE | FALSE | sp \| RETBP_HUMAN \| | sp \| RETBP_HUMAN \| | 23 kDa | | 4 |
| 270 | TRUE | FALSE | Uncharacterized protein | tr \| F6YC23 \| | 14 kDa | | 2 |
| | | | OS = Equus caballus | F6YC23_HORSE | | | |
| | | | GN = RPL31 PE = 4 SV = 1 | | | | |
| 271 | TRUE | FALSE | Uncharacterized protein | tr \| F6WLX9 \| | 55 kDa | | 1 |
| | | | OS = Equus caballus | F6WLX9_HORSE | | | |
| | | | GN = C22orf28 PE = 4 SV = 1 | | | | |
| 272 | TRUE | FALSE | Uncharacterized protein | tr \| F7DPW3 \| | 36 kDa | | 3 |
| | | | OS = Equus caballus | F7DPW3_HORSE | | | |
| | | | GN = SLC25A31 PE = 3 SV = 1 | | | | |
| 273 | TRUE | FALSE | Uncharacterized protein | tr \| F7D9J2 \| | 65 kDa | | 2 |
| | | | (Fragment) | F7D9J2_HORSE | | | |
| | | | OS = Equus caballus | | | | |
| | | | GN = TKT PE = 4 SV = 1 | | | | |
| 274 | TRUE | FALSE | Uncharacterized protein | tr \| F6WM79 \| | 45 kDa | | 3 |
| | | | OS = Equus caballus | F6WM79_HORSE | | | |
| | | | GN = ACTR2 PE = 3 SV = 1 | | | | |
| 275 | TRUE | FALSE | Uncharacterized protein | tr \| F7C4D4 \| | 107 kDa | | 3 |
| | | | OS = Equus caballus | F7C4D4_HORSE | | | |
| | | | GN = GANAB PE = 4 SV = 1 | (+1) | | | |
| 276 | TRUE | FALSE | Uncharacterized protein | tr \| F6PSX1 \| | 45 kDa | | 3 |
| | | | OS = Equus caballus | F6PSX1_HORSE | | | |
| | | | GN = RCC1 PE = 4 SV = 1 | | | | |
| 277 | TRUE | FALSE | Uncharacterized protein | tr \| F6R1X9 \| | 42 kDa | TRUE | 3 |
| | | | OS = Equus caballus | F6R1X9_HORSE | | | |
| | | | GN = ACTC1 PE = 3 SV = 1 | | | | |
| 278 | TRUE | FALSE | Uncharacterized protein | tr \| F6R3A3 \| | 25 kDa | | 4 |
| | | | (Fragment) | F6R3A3_HORSE | | | |
| | | | OS = Equus caballus | | | | |
| | | | GN = EIF4H PE = 4 SV = 1 | | | | |
| 279 | TRUE | FALSE | Uncharacterized protein | tr \| F6ULJ8 \| | 16 kDa | | 4 |
| | | | OS = Equus caballus | F6ULJ8_HORSE | | | |
| | | | GN = CRABP1 PE = 3 SV = 1 | | | | |
| 280 | TRUE | FALSE | Uncharacterized protein | tr \| F6Z049 \| | 32 kDa | | 4 |
| | | | OS = Equus caballus | F6Z049_HORSE | | | |
| | | | GN = MLEC PE = 4 SV = 1 | | | | |
| 281 | TRUE | FALSE | Uncharacterized protein | tr \| F6ZER8 \| | 27 kDa | | 4 |
| | | | OS = Equus caballus | F6ZER8_HORSE | | | |
| | | | GN = REXO2 PE = 3 SV = 1 | | | | |
| 282 | TRUE | FALSE | Uncharacterized protein | tr \| F7CEE6 \| | 36 kDa | | 2 |
| | | | OS = Equus caballus | F7CEE6_HORSE | | | |
| | | | GN = DHRS9 PE = 3 SV = 1 | | | | |
| 283 | TRUE | FALSE | Uncharacterized protein | tr \| F7D6B3 \| | 103 kDa | | 4 |
| | | | OS = Equus caballus | F7D6B3_HORSE | | | |
| | | | GN = RRBP1 PE = 4 SV = 1 | (+2) | | | |
| 284 | TRUE | FALSE | Uncharacterized protein | tr \| F7E1Q5 \| | 45 kDa | | 3 |
| | | | OS = Equus caballus | F7E1Q5_HORSE | | | |
| | | | GN = PPP1R32 PE = 4 SV = 1 | | | | |
| 285 | TRUE | FALSE | Annexin | tr \| F7E419 \| | 37 kDa | | 4 |
| | | | OS = Equus caballus | F7E419_HORSE | | | |
| | | | GN = ANXA8L1 PE = 3 SV = 1 | | | | |
| 286 | TRUE | FALSE | Uncharacterized protein | tr \| F6WWW7 \| | 31 kDa | TRUE | 3 |
| | | | (Fragment) | F6WWW7_HORSE | | | |
| | | | OS = Equus caballus | | | | |
| | | | GN = CAPZA1 PE = 4 SV = 1 | | | | |
| 287 | TRUE | FALSE | Uncharacterized protein | tr \| F7BXM5 \| | 49 kDa | | 3 |
| | | | (Fragment) | F7BXM5_HORSE | | | |
| | | | OS = Equus caballus | | | | |
| | | | GN = UQCRC1 PE = 3 SV = 1 | | | | |
| 288 | TRUE | FALSE | Uncharacterized protein | tr \| F6WHS3 \| | 34 kDa | | 3 |
| | | | (Fragment) | F6WHS3_HORSE | | | |
| | | | OS = Equus caballus | | | | |
| | | | GN = HNRNPD PE = 4 SV = 1 | | | | |
| 289 | TRUE | FALSE | Uncharacterized protein | tr \| F6PTX3 \| | 13 kDa | | 3 |
| | | | OS = Equus caballus | F6PTX3_HORSE | | | |
| | | | GN = RPL35A PE = 4 SV = 1 | (+1) | | | |
| 290 | TRUE | FALSE | Uncharacterized protein | tr \| F6YBT5 \| | 68 kDa | | 1 |
| | | | OS = Equus caballus | F6YBT5_HORSE | | | |
| | | | GN = ATP6V1A PE = 3 SV = 1 | | | | |
| 291 | TRUE | FALSE | Uncharacterized protein | tr \| F7BXA6 \| | 91 kDa | | 2 |
| | | | (Fragment) | F7BXA6_HORSE | | | |
| | | | OS = Equus caballus | | | | |
| | | | GN = NPEPPS PE = 4 SV = 1 | | | | |
| 292 | TRUE | FALSE | Uncharacterized protein | tr \| F6ZIY3 \| | 67 kDa | | 2 |
| | | | (Fragment) | F6ZIY3_HORSE | | | |
| | | | OS = Equus caballus | | | | |
| | | | GN = SWAP70 PE = 4 SV = 1 | | | | |
| 293 | TRUE | FALSE | Uncharacterized protein | tr \| F6QR51 \| | 14 kDa | | 2 |
| | | | OS = Equus caballus | F6QR51_HORSE | | | |
| | | | GN = SUB1 PE = 4 SV = 1 | | | | |
| 294 | TRUE | FALSE | Uncharacterized protein | tr \| F6R6I2 \| | 82 kDa | | 1 |
| | | | OS = Equus caballus | F6R6I2_HORSE | | | |
| | | | GN = IMMT PE = 4 SV = 1 | (+1) | | | |
| 295 | TRUE | FALSE | Uncharacterized protein | tr \| F6TD06 \| | 21 kDa | | 3 |
| | | | OS = Equus caballus | F6TD06_HORSE | | | |
| | | | GN = ARF3 PE = 3 SV = 1 | | | | |
| 296 | TRUE | FALSE | Uncharacterized protein | tr \| F6VX41 \| | 49 kDa | | 3 |
| | | | (Fragment) | F6VX41_HORSE | | | |
| | | | OS = Equus caballus | | | | |
| | | | GN = RPL4 PE = 4 SV = 1 | | | | |
| 297 | TRUE | FALSE | Proteasome subunit alpha type | tr \| F6WZW6 \| | 30 kDa | | 3 |
| | | | OS = Equus caballus | F6WZW6_HORSE | | | |
| | | | GN = PSMA1 PE = 3 | | | | |
| 298 | TRUE | FALSE | Uncharacterized protein | tr \| F6X7G5 \| | 46 kDa | | 1 |
| | | | OS = Equus caballus | F6X7G5_HORSE | | | |
| | | | GN = HNRNPF PE = 4 SV = 1 | | | | |
| 299 | TRUE | FALSE | Uncharacterized protein | tr \| F6XA04 \| | 27 kDa | | 3 |
| | | | OS = Equus caballus | F6XA04_HORSE | | | |
| | | | GN = YWHAE PE = 3 SV = 1 | | | | |
| 300 | TRUE | FALSE | Uncharacterized protein | tr \| F6YZX2 \| | 17 kDa | | 3 |
| | | | (Fragment) | F6YZX2_HORSE | | | |
| | | | OS = Equus caballus | | | | |
| | | | GN = RPS19 PE = 4 SV = 1 | | | | |
| 301 | TRUE | FALSE | Uncharacterized protein | tr \| F6ZDJ2 \| | 202 kDa | | 3 |
| | | | OS = Equus caballus | F6ZDJ2_HORSE | | | |
| | | | GN = LAMA4 PE = 4 SV = 1 | (+1) | | | |
| 302 | TRUE | FALSE | Uncharacterized protein | tr \| F7AKK7 \| | 36 kDa | | 3 |
| | | | (Fragment) | F7AKK7_HORSE | | | |
| | | | OS = Equus caballus | | | | |
| | | | GN = EIF3I PE = 4 SV = 1 | | | | |
| 303 | TRUE | FALSE | T-complex protein 1 subunit | tr \| F7AX81 \| | 60 kDa | | 3 |
| | | | alpha (Fragment) | F7AX81_HORSE | | | |
| | | | OS = Equus caballus | | | | |
| | | | GN = TCP1 PE = 3 SV = 1 | | | | |
| 304 | TRUE | FALSE | Uncharacterized protein | tr \| F7B2B9 \| | 120 kDa | | 3 |
| | | | (Fragment) | F7B2B9_HORSE | | | |
| | | | OS = Equus caballus | (+1) | | | |
| | | | GN = MYO1B PE = 4 SV = 1 | | | | |
| 305 | TRUE | FALSE | Uncharacterized protein | tr \| F7B3L3 \| | 16 kDa | | 3 |
| | | | (Fragment) | F7B3L3_HORSE | | | |
| | | | OS = Equus caballus | | | | |
| | | | GN = RPL27A PE = 3 SV = 1 | | | | |
| 306 | TRUE | FALSE | Uncharacterized protein | tr \| F7B8E7 \| | 85 kDa | | 3 |
| | | | OS = Equus caballus | F7B8E7_HORSE | | | |
| | | | GN = MME PE = 4 SV = 1 | | | | |
| 307 | TRUE | FALSE | Uncharacterized protein | tr \| F7BPE3 \| | 18 kDa | | 3 |
| | | | (Fragment) | F7BPE3_HORSE | | | |
| | | | OS = Equus caballus | | | | |
| | | | GN = ALYREF PE = 4 SV = 1 | | | | |
| 308 | TRUE | FALSE | Uncharacterized protein | tr \| F7BWU2 \| | 19 kDa | | 3 |
| | | | OS = Equus caballus | F7BWU2_HORSE | | | |
| | | | GN = AP1S1 PE = 4 SV = 1 | | | | |
| 309 | TRUE | FALSE | Annexin | tr \| F6W787 \| | 72 kDa | | 1 |
| | | | OS = Equus caballus | F6W787_HORSE | | | |
| | | | GN = ANXA6 PE = 3 SV = 1 | | | | |
| 310 | TRUE | FALSE | Glucose-6-phosphate 1- | tr \| F7DMG5 \| | 59 kDa | | 1 |
| | | | dehydrogenase | F7DMG5_HORSE | | | |
| | | | OS = Equus caballus | | | | |
| | | | GN = G6PD PE = 3 SV = 1 | | | | |
| 311 | TRUE | FALSE | Uncharacterized protein | tr \| F7BEV6 \| | 108 kDa | | 3 |
| | | | (Fragment) | F7BEV6_HORSE | | | |
| | | | OS = Equus caballus | | | | |
| | | | GN = MOV10 PE = 4 SV = 1 | | | | |
| 312 | TRUE | FALSE | Histone H2B | tr \| F6UGW9 \| | 14 kDa | TRUE | 3 |
| | | | OS = Equus caballus | F6UGW9_HORSE | | | |
| | | | GN = HIST1H2BJ PE = 3 SV = 1 | | | | |
| 313 | TRUE | FALSE | Uncharacterized protein | tr \| F6XZY5 \| | 15 kDa | | 3 |
| | | | OS = Equus caballus | F6XZY5_HORSE | | | |
| | | | GN = RPL35 PE = 3 SV = 1 | | | | |
| 314 | TRUE | FALSE | Uncharacterized protein | tr \| F6ULU9 \| | 18 kDa | | 2 |
| | | | OS = Equus caballus | F6ULU9_HORSE | | | |
| | | | GN = LOC100072076 PE = 3 SV = 1 | | | | |
| 315 | TRUE | FALSE | Uncharacterized protein | tr \| F7C373 \| | 55 kDa | | 3 |
| | | | (Fragment) | F7C373_HORSE | | | |
| | | | OS = Equus caballus | | | | |
| | | | GN = HSPA9 PE = 3 SV = 1 | | | | |
| 316 | TRUE | FALSE | 60S ribosomal protein L6 | tr \| F6QF58 \| | 30 kDa | | 2 |
| | | | (Fragment) | F6QF58_HORSE | | | |
| | | | OS = Equus caballus | (+1) | | | |
| | | | GN = LOC100051509 PE = 3 SV = 1 | | | | |
| 317 | TRUE | FALSE | Proteasome subunit alpha type | tr \| F7BRA4 \| | 27 kDa | | 2 |
| | | | OS = Equus caballus | F7BRA4_HORSE | | | |
| | | | GN = PSMA6 PE = 3 | | | | |
| 318 | TRUE | FALSE | Uncharacterized protein | tr \| F7DCS5 \| | 101 kDa | | 2 |
| | | | (Fragment) | F7DCS5_HORSE | | | |
| | | | OS = Equus caballus | (+1) | | | |
| | | | GN = AP2A1 PE = 4 SV = 1 | | | | |
| 319 | TRUE | FALSE | Uncharacterized protein | tr \| F6PTY9 \| | 19 kDa | | 2 |
| | | | OS = Equus caballus | F6PTY9_HORSE | | | |
| | | | GN = LOC100061551 PE = 4 SV = 1 | (+3) | | | |
| 320 | TRUE | FALSE | Uncharacterized protein | tr \| F7AX30 \| | 21 kDa | TRUE | 1 |
| | | | OS = Equus caballus | F7AX30_HORSE | | | |
| | | | GN = RAC2 PE = 3 SV = 1 | | | | |
| 321 | TRUE | FALSE | Uncharacterized protein | REVERSE_tr \| | 63 kDa | | 3 |
| | | | OS = Equus caballus | F6WGX6 \| | | | |
| | | | GN = KRT77 PE = 3 SV = 1 | F6WGX6_HORSE-R | | | |
| 322 | TRUE | FALSE | sp \| TRYP_PIG \| | sp \| TRYP_PIG \| | 24 kDa | | 3 |
| 323 | TRUE | FALSE | Uncharacterized protein | tr \| F6PH57 \| | 20 kDa | | 3 |
| | | | OS = Equus caballus | F6PH57_HORSE | | | |
| | | | GN = ARPC4 PE = 4 SV = 1 | | | | |
| 324 | TRUE | FALSE | Uncharacterized protein | tr \| F6PWV8 \| | 46 kDa | | 3 |
| | | | OS = Equus caballus | F6PWV8_HORSE | | | |
| | | | GN = SERPINH1 PE = 3 SV = 1 | | | | |
| 325 | TRUE | FALSE | Uncharacterized protein | tr \| F6S0P5 \| | 21 kDa | | 3 |
| | | | OS = Equus caballus | F6S0P5_HORSE | | | |
| | | | GN = ARF4 PE = 3 SV = 1 | | | | |
| 326 | TRUE | FALSE | Uncharacterized protein | tr \| F6T937 \| | 11 kDa | | 3 |
| | | | (Fragment) | F6T937_HORSE | | | |
| | | | OS = Equus caballus | (+2) | | | |
| | | | GN = SUMO3 PE = 4 SV = 1 | | | | |
| 327 | TRUE | FALSE | Uncharacterized protein | tr \| F6TQ92 \| | 66 kDa | | 3 |
| | | | (Fragment) | F6TQ92_HORSE | | | |
| | | | OS = Equus caballus | | | | |
| | | | GN = PABPC1 PE = 4 SV = 1 | | | | |
| 328 | TRUE | FALSE | Uncharacterized protein | tr \| F6TXV7 \| | 54 kDa | | 3 |
| | | | (Fragment) | F6TXV7_HORSE | | | |
| | | | OS = Equus caballus | | | | |
| | | | GN = SLC39A14 PE = 4 SV = 1 | | | | |
| 329 | TRUE | FALSE | Uncharacterized protein | tr \| F6UCU8 \| | 58 kDa | TRUE | 3 |
| | | | (Fragment) | F6UCU8_HORSE | | | |
| | | | OS = Equus caballus | | | | |
| | | | GN = LMNB1 PE = 3 SV = 1 | | | | |
| 330 | TRUE | FALSE | Uncharacterized protein | tr \| F6UFV3 \| | 41 kDa | | 3 |
| | | | OS = Equus caballus | F6UFV3_HORSE | | | |
| | | | GN = ADK PE = 4 SV = 1 | | | | |
| 331 | TRUE | FALSE | 40S ribosomal protein S12 | tr \| F6VGG1 \| | 15 kDa | | 3 |
| | | | OS = Equus caballus | F6VGG1_HORSE | | | |
| | | | GN = RPS12 PE = 3 SV = 1 | (+1) | | | |
| 332 | TRUE | FALSE | Uncharacterized protein | tr \| F6VIV2 \| | 14 kDa | | 3 |
| | | | OS = Equus caballus | F6VIV2_HORSE | | | |
| | | | GN = HINT1 PE = 4 SV = 1 | | | | |
| 333 | TRUE | FALSE | Uncharacterized protein | tr \| F6W2Y1 \| | 50 kDa | | 3 |
| | | | OS = Equus caballus | F6W2Y1_HORSE | | | |
| | | | GN = FGG PE = 4 SV = 1 | | | | |
| 334 | TRUE | FALSE | Uncharacterized protein | tr \| F6WPB4 \| | 20 kDa | | 3 |
| | | | OS = Equus caballus | F6WPB4_HORSE | | | |
| | | | GN = PARK7 PE = 4 SV = 1 | | | | |
| 335 | TRUE | FALSE | Uncharacterized protein | tr \| F6WQI9 \| | 60 kDa | | 2 |
| | | | (Fragment) | F6WQI9_HORSE | | | |
| | | | OS = Equus caballus | | | | |
| | | | GN = RPN1 PE = 4 SV = 1 | | | | |
| 336 | TRUE | FALSE | Uncharacterized protein | tr \| F6WQM7 \| | 43 kDa | | 2 |
| | | | OS = Equus caballus | F6WQM7_HORSE | | | |
| | | | GN = PDHA1 PE = 4 SV = 1 | | | | |
| 337 | TRUE | FALSE | Uncharacterized protein | tr \| F6WUU4 \| | 26 kDa | | 3 |
| | | | OS = Equus caballus | F6WUU4_HORSE | | | |
| | | | GN = ATP6V1E1 PE = 3 SV = 1 | | | | |
| 338 | TRUE | FALSE | Uncharacterized protein | tr \| F6XSN2 \| | 59 kDa | | 3 |
| | | | (Fragment) | F6XSN2_HORSE | | | |
| | | | OS = Equus caballus | | | | |
| | | | GN = CCT7 PE = 3 SV = 1 | | | | |
| 339 | TRUE | FALSE | Uncharacterized protein | tr \| F6ZQ56 \| | 19 kDa | | 3 |
| | | | OS = Equus caballus | F6ZQ56_HORSE | | | |
| | | | GN = PTGES3 PE = 4 SV = 1 | | | | |
| 340 | TRUE | FALSE | Uncharacterized protein | tr \| F7BC94 \| | 109 kDa | | 3 |
| | | | OS = Equus caballus | F7BC94_HORSE | | | |
| | | | GN = HYOU1 PE = 3 SV = 1 | | | | |
| 341 | TRUE | FALSE | Uncharacterized protein | tr \| F7CTS5 \| | 36 kDa | | 3 |
| | | | OS = Equus caballus | F7CTS5_HORSE | | | |
| | | | GN = EIF2S1 PE = 4 SV = 1 | | | | |
| 342 | TRUE | FALSE | Uncharacterized protein | tr \| F7D8L9 \| | 47 kDa | | 2 |
| | | | OS = Equus caballus | F7D8L9_HORSE | | | |
| | | | GN = ASS1 PE = 3 SV = 1 | | | | |
| 343 | TRUE | FALSE | Annexin | tr \| F7DE06 \| | 54 kDa | | 3 |
| | | | OS = Equus caballus | F7DE06_HORSE | | | |
| | | | GN = ANXA11 PE = 3 SV = 1 | | | | |
| 344 | TRUE | FALSE | Uncharacterized protein | tr \| F7DL64 \| | 29 kDa | | 3 |
| | | | (Fragment) | F7DL64_HORSE | | | |
| | | | OS = Equus caballus | | | | |
| | | | GN = HNRNPAB PE = 4 SV = 1 | | | | |
| 345 | TRUE | FALSE | Uncharacterized protein | tr \| H9GZN9 \| | 49 kDa | | 3 |
| | | | (Fragment) | H9GZN9_HORSE | | | |
| | | | OS = Equus caballus | | | | |
| | | | GN = IGHM PE = 4 SV = 1 | | | | |
| 346 | TRUE | FALSE | Glyceraldehyde-3-phosphate | tr \| Q0QET0 \| | 24 kDa | TRUE | 2 |
| | | | dehydrogenase (Fragment) | Q0QET0_HORSE | | | |
| | | | OS = Equus caballus | | | | |
| | | | GN = GAPDH PE = 2 | | | | |
| 347 | TRUE | FALSE | Uncharacterized protein | REVERSE_tr \| | 207 kDa | | 1 |
| | | | OS = Equus caballus | F6YYD2 \| | | | |
| | | | GN = GBF1 PE = 4 SV = 1 | F6YYD2_HORSE-R | | | |
| 348 | TRUE | FALSE | 60S ribosomal protein L13 | tr \| F6ZX61 \| | 24 kDa | | 2 |
| | | | OS = Equus caballus | F6ZX61_HORSE | | | |
| | | | GN = RPL13 PE = 3 SV = 1 | | | | |
| 349 | TRUE | FALSE | Uncharacterized protein | tr \| F6S1I3 \| | 43 kDa | | 2 |
| | | | (Fragment) | F6S1I3_HORSE | | | |
| | | | OS = Equus caballus | (+2) | | | |
| | | | GN = ACADS PE = 3 SV = 1 | | | | |
| 350 | TRUE | FALSE | Uncharacterized protein | tr \| F6YX05 \| | 229 kDa | TRUE | 2 |
| | | | OS = Equus caballus | F6YX05_HORSE | | | |
| | | | GN = MYH10 PE = 4 SV = 1 | | | | |
| 351 | TRUE | FALSE | Uncharacterized protein | tr \| F7AYR2 \| | 25 kDa | | 2 |
| | | | OS = Equus caballus | F7AYR2_HORSE | | | |
| | | | GN = RPL10 PE = 4 SV = 1 | | | | |
| 352 | TRUE | FALSE | Uncharacterized protein | tr \| F7BTD8 \| | 311 kDa | TRUE | 2 |
| | | | OS = Equus caballus | F7BTD8_HORSE | | | |
| | | | GN = FBN1 PE = 4 SV = 1 | (+1) | | | |
| 353 | TRUE | FALSE | Uncharacterized protein | tr \| F7CIM1 \| | 28 kDa | TRUE | 2 |
| | | | OS = Equus caballus | F7CIM1_HORSE | | | |
| | | | GN = SFN PE = 3 SV = 1 | | | | |
| 354 | TRUE | FALSE | Uncharacterized protein | tr \| F7D9I5 \| | 53 kDa | | 2 |
| | | | OS = Equus caballus | F7D9I5_HORSE | | | |
| | | | GN = EPHX1 PE = 4 SV = 1 | | | | |
| 355 | TRUE | FALSE | Uncharacterized protein | tr \| F6RX11 \| | 13 kDa | | 2 |
| | | | OS = Equus caballus | F6RX11_HORSE | | | |
| | | | GN = RPS20 PE = 4 SV = 1 | | | | |
| 356 | TRUE | FALSE | Uncharacterized protein | tr \| F6RC09 \| | 92 kDa | | 2 |
| | | | (Fragment) | F6RC09_HORSE | | | |
| | | | OS = Equus caballus | | | | |
| | | | GN = VPS35 PE = 4 SV = 1 | | | | |
| 357 | TRUE | FALSE | Uncharacterized protein | tr \| F6YGF7 \| | 611 kDa | | 2 |
| | | | (Fragment) | F6YGF7_HORSE | | | |
| | | | OS = Equus caballus | (+1) | | | |
| | | | GN = MACF1 PE = 4 SV = 1 | | | | |
| 358 | TRUE | FALSE | Uncharacterized protein | tr \| F6QW38 \| | 35 kDa | | 2 |
| | | | (Fragment) | F6QW38_HORSE | | | |
| | | | OS = Equus caballus | | | | |
| | | | GN = MTHFD2 PE = 3 SV = 1 | | | | |
| 359 | TRUE | FALSE | Uncharacterized protein | tr \| F7AZ73 \| | 66 kDa | | 2 |
| | | | (Fragment) | F7AZ73_HORSE | | | |
| | | | OS = Equus caballus | | | | |
| | | | GN = STXBP2 PE = 4 SV = 1 | | | | |
| 360 | TRUE | FALSE | Uncharacterized protein | tr \| F6XTN1 \| | 46 kDa | | 1 |
| | | | OS = Equus caballus | F6XTN1_HORSE | | | |
| | | | GN = EIF4A1 PE = 3 SV = 1 | | | | |
| 361 | TRUE | FALSE | Uncharacterized protein | tr \| F6VM62 \| | 27 kDa | | 1 |
| | | | OS = Equus caballus | F6VM62_HORSE | | | |
| | | | GN = LOC100068443 PE = 3 SV = 1 | | | | |
| 362 | TRUE | FALSE | Uncharacterized protein | tr \| F7BAL2 \| | 22 kDa | | 1 |
| | | | (Fragment) | F7BAL2_HORSE | | | |
| | | | OS = Equus caballus | | | | |
| | | | GN = RPL17 PE = 3 SV = 1 | | | | |
| 363 | TRUE | FALSE | Uncharacterized protein | tr \| F7BTW7 \| | 186 kDa | | 1 |
| | | | OS = Equus caballus | F7BTW7_HORSE | | | |
| | | | GN = LOC100060539 PE = 4 SV = 1 | (+2) | | | |
| 364 | TRUE | FALSE | GMP reductase | tr \| F7DHY2 \| | 38 kDa | | 1 |
| | | | OS = Equus caballus | F7DHY2_HORSE | | | |
| | | | GN = GMPR2 PE = 3 SV = 1 | | | | |
| 365 | TRUE | FALSE | Uncharacterized protein | tr \| F6UJV4 \| | 87 kDa | | 1 |
| | | | OS = Equus caballus | F6UJV4_HORSE | | | |
| | | | GN = DHX15 PE = 4 SV = 1 | (+1) | | | |
| 366 | TRUE | FALSE | 60S ribosomal protein L27 | tr \| F7D8K7 \| | 16 kDa | | 1 |
| | | | OS = Equus caballus | F7D8K7_HORSE | | | |
| | | | GN = RPL27 PE = 3 SV = 1 | | | | |
| 367 | TRUE | FALSE | Uncharacterized protein | tr \| F6SN52 \| | 18 kDa | | 1 |
| | | | (Fragment) | F6SN52_HORSE | | | |
| | | | OS = Equus caballus | (+2) | | | |
| | | | GN = EIF5A PE = 4 SV = 1 | | | | |
| 368 | TRUE | FALSE | Uncharacterized protein | tr \| F7B812 \| | 35 kDa | | 1 |
| | | | (Fragment) | F7B812_HORSE | | | |
| | | | OS = Equus caballus | | | | |
| | | | GN = HTRA1 PE = 4 SV = 1 | | | | |
| 369 | TRUE | FALSE | Uncharacterized protein | REVERSE_tr \| | 176 kDa | | 1 |
| | | | OS = Equus caballus | F7AZ01 \| | | | |
| | | | GN = ZNF831 PE = 4 SV = 1 | F7AZ01_HORSE-R | | | |
| 370 | TRUE | FALSE | Uncharacterized protein | REVERSE_tr \| | 77 kDa | | 1 |
| | | | OS = Equus caballus | F6SPL7 \| | | | |
| | | | GN = C10orf2 PE = 4 SV = 1 | F6SPL7_HORSE-R | | | |
| | | | | (+1) | | | |
| 371 | TRUE | FALSE | sp \| CYB5_HUMAN \| | sp \| CYB5_HUMAN \| | 15 kDa | | 2 |
| 372 | TRUE | FALSE | Pigment epithelium-derived | tr \| B8K250 \| | 46 kDa | | 2 |
| | | | factor | B8K250_HORSE | | | |
| | | | OS = Equus caballus | (+1) | | | |
| | | | PE = 2 SV = 1 | | | | |
| 373 | TRUE | FALSE | Clathrin light chain | tr \| C0SW05 \| | 23 kDa | | 2 |
| | | | OS = Equus caballus | C0SW05_HORSE | | | |
| | | | GN = clc PE = 2 SV = 1 | | | | |
| 374 | TRUE | FALSE | Uncharacterized protein | tr \| F6PQP4 \| | 14 kDa | | 1 |
| | | | OS = Equus caballus | F6PQP4_HORSE | | | |
| | | | GN = C11orf52 PE = 4 SV = 1 | | | | |
| 375 | TRUE | FALSE | Uncharacterized protein | tr \| F6PQT9 \| | 31 kDa | | 2 |
| | | | (Fragment) | F6PQT9_HORSE | | | |
| | | | OS = Equus caballus | | | | |
| | | | GN = VDAC1 PE = 4 SV = 1 | | | | |
| 376 | TRUE | FALSE | Uncharacterized protein | tr \| F6QD46 \| | 45 kDa | | 2 |
| | | | (Fragment) | F6QD46_HORSE | | | |
| | | | OS = Equus caballus | | | | |
| | | | GN = SNX6 PE = 4 SV = 1 | | | | |
| 377 | TRUE | FALSE | Uncharacterized protein | tr \| F6QLX7 \| | 115 kDa | | 2 |
| | | | OS = Equus caballus | F6QLX7_HORSE | | | |
| | | | GN = ITGA5 PE = 3 SV = 1 | (+1) | | | |
| 378 | TRUE | FALSE | Eukaryotic translation initiation | tr \| F6R0W4 \| | 26 kDa | | 2 |
| | | | factor 6 | F6R0W4_HORSE | | | |
| | | | OS = Equus caballus | | | | |
| | | | GN = EIF6 PE = 3 SV = 1 | | | | |
| 379 | TRUE | FALSE | Eukaryotic translation initiation | tr \| F6R1D6 \| | 52 kDa | | 2 |
| | | | factor 3 subunit E | F6R1D6_HORSE | | | |
| | | | OS = Equus caballus | | | | |
| | | | GN = EIF3E PE = 3 SV = 1 | | | | |
| 380 | TRUE | FALSE | Uncharacterized protein | tr \| F6RCA8 \| | 17 kDa | | 1 |
| | | | (Fragment) | F6RCA8_HORSE | | | |
| | | | OS = Equus caballus | | | | |
| | | | GN = PRDX5 PE = 4 SV = 1 | | | | |
| 381 | TRUE | FALSE | Uncharacterized protein | tr \| F6RMW9 \| | 55 kDa | | 2 |
| | | | OS = Equus caballus | F6RMW9_HORSE | | | |
| | | | GN = LOC100053371 PE = 3 SV = 1 | | | | |
| 382 | TRUE | FALSE | Uncharacterized protein | tr \| F6RV24 \| | 63 kDa | | 2 |
| | | | OS = Equus caballus | F6RV24_HORSE | | | |
| | | | GN = IGF2BP1 PE = 4 SV = 1 | | | | |
| 383 | TRUE | FALSE | Glutathione S-transferase | tr \| F6RZ56 \| | 25 kDa | | 2 |
| | | | OS = Equus caballus | F6RZ56_HORSE | | | |
| | | | GN = LOC100056506 PE = 3 SV = 1 | (+4) | | | |
| 384 | TRUE | FALSE | Uncharacterized protein | tr \| F6T161 \| | 32 kDa | | 2 |
| | | | OS = Equus caballus | F6T161_HORSE | | | |
| | | | GN = ARGLU1 PE = 4 SV = 1 | | | | |
| 385 | TRUE | FALSE | Uncharacterized protein | tr \| F6TBN7 \| | 48 kDa | | 2 |
| | | | (Fragment) | F6TBN7_HORSE | | | |
| | | | OS = Equus caballus | | | | |
| | | | GN = FKBP4 PE = 4 SV = 1 | | | | |
| 386 | TRUE | FALSE | Uncharacterized protein | tr \| F6TJX5 \| | 62 kDa | | 2 |
| | | | OS = Equus caballus | F6TJX5_HORSE | | | |
| | | | GN = TPP1 PE = 4 SV = 1 | | | | |
| 387 | TRUE | FALSE | Uncharacterized protein | tr \| F6TMH1 \| | 47 kDa | | 1 |
| | | | (Fragment) | F6TMH1_HORSE | | | |
| | | | OS = Equus caballus | | | | |
| | | | GN = COPS4 PE = 4 SV = 1 | | | | |
| 388 | TRUE | FALSE | Uncharacterized protein | tr \| F6TRQ6 \| | 78 kDa | | 2 |
| | | | (Fragment) | F6TRQ6_HORSE | | | |
| | | | OS = Equus caballus | | | | |
| | | | PE = 4 SV = 1 | | | | |
| 389 | TRUE | FALSE | Uncharacterized protein | tr \| F6U2A1 \| | 57 kDa | | 2 |
| | | | OS = Equus caballus | F6U2A1_HORSE | | | |
| | | | GN = API5 PE = 4 SV = 1 | | | | |
| 390 | TRUE | FALSE | Uncharacterized protein | tr \| F6U6K1 \| | 41 kDa | | 2 |
| | | | OS = Equus caballus | F6U6K1_HORSE | | | |
| | | | GN = SUCLG2 PE = 3 SV = 1 | | | | |
| 391 | TRUE | FALSE | Uncharacterized protein | tr \| F6U974 \| | 10 kDa | | 2 |
| | | | (Fragment) | F6U974_HORSE | | | |
| | | | OS = Equus caballus | (+1) | | | |
| | | | GN = LOC100050696 PE = 4 SV = 1 | | | | |
| 392 | TRUE | FALSE | Uncharacterized protein | tr \| F6UF75 \| | 12 kDa | | 2 |
| | | | OS = Equus caballus | F6UF75_HORSE | | | |
| | | | GN = S100A4 PE = 4 SV = 1 | | | | |
| 393 | TRUE | FALSE | Uncharacterized protein | tr \| F6UVF8 \| | 48 kDa | | 2 |
| | | | (Fragment) | F6UVF8_HORSE | | | |
| | | | OS = Equus caballus | | | | |
| | | | GN = SEPT7 PE = 3 SV = 1 | | | | |
| 394 | TRUE | FALSE | Uncharacterized protein | tr \| F6UYF5 \| | 136 kDa | | 2 |
| | | | OS = Equus caballus | F6UYF5_HORSE | | | |
| | | | GN = CGN PE = 4 SV = 1 | | | | |
| 395 | TRUE | FALSE | Uncharacterized protein | tr \| F6VPC9 \| | 20 kDa | | 1 |
| | | | OS = Equus caballus | F6VPC9_HORSE | | | |
| | | | GN = MFAP2 PE = 4 SV = 1 | | | | |
| 396 | TRUE | FALSE | Uncharacterized protein | tr \| F6VSN2 \| | 23 kDa | | 2 |
| | | | OS = Equus caballus | F6VSN2_HORSE | | | |
| | | | GN = LOC100053249 PE = 3 SV = 1 | | | | |
| 397 | TRUE | FALSE | Uncharacterized protein | tr \| F6VUI6 \| | 109 kDa | TRUE | 2 |
| | | | OS = Equus caballus | F6VUI6_HORSE | | | |
| | | | GN = EFTUD2 PE = 4 SV = 1 | | | | |
| 398 | TRUE | FALSE | Proteasome subunit beta type | tr \| F6W2I4 \| | 25 kDa | | 2 |
| | | | OS = Equus caballus | F6W2I4_HORSE | | | |
| | | | GN = PSMB6 PE = 3 SV = 1 | | | | |
| 399 | TRUE | FALSE | Uncharacterized protein | tr \| F6WSE2 \| | 116 kDa | | 2 |
| | | | OS = Equus caballus | F6WSE2_HORSE | | | |
| | | | GN = OGDH PE = 4 SV = 1 | (+2) | | | |
| 400 | TRUE | FALSE | Uncharacterized protein | tr \| F6X0P8 \| | 105 kDa | | 1 |
| | | | (Fragment) | F6X0P8_HORSE | | | |
| | | | OS = Equus caballus | | | | |
| | | | GN = KIF5B PE = 3 SV = 1 | | | | |
| 401 | TRUE | FALSE | Uncharacterized protein | tr \| F6XEP5 \| | 51 kDa | | 2 |
| | | | (Fragment) | F6XEP5_HORSE | | | |
| | | | OS = Equus caballus | | | | |
| | | | GN = NT5E PE = 4 SV = 1 | | | | |
| 402 | TRUE | FALSE | Uncharacterized protein | tr \| F6XEW8 \| | 20 kDa | | 2 |
| | | | OS = Equus caballus | F6XEW8_HORSE | | | |
| | | | GN = RBM8A PE = 4 SV = 1 | | | | |
| 403 | TRUE | FALSE | Uncharacterized protein | tr \| F6YAH4 \| | 129 kDa | | 2 |
| | | | OS = Equus caballus | F6YAH4_HORSE | | | |
| | | | GN = ANKFY1 PE = 4 SV = 1 | | | | |
| 404 | TRUE | FALSE | Uncharacterized protein | tr \| F6YGW2 \| | 19 kDa | | 2 |
| | | | OS = Equus caballus | F6YGW2_HORSE | | | |
| | | | GN = BAX PE = 4 SV = 1 | | | | |
| 405 | TRUE | FALSE | Histone H3 | tr \| F6YXV8 \| | 15 kDa | TRUE | 2 |
| | | | OS = Equus caballus | F6YXV8_HORSE | | | |
| | | | GN = LOC100054462 PE = 3 SV = 1 | | | | |
| 406 | TRUE | FALSE | Uncharacterized protein | tr \| F6Z8Y3 \| | 65 kDa | | 2 |
| | | | (Fragment) | F6Z8Y3_HORSE | | | |
| | | | OS = Equus caballus | | | | |
| | | | GN = BCAM PE = 4 SV = 1 | | | | |
| 407 | TRUE | FALSE | Uncharacterized protein | tr \| F6ZB53 \| | 35 kDa | | 2 |
| | | | OS = Equus caballus | F6ZB53_HORSE | | | |
| | | | GN = APEX1 PE = 4 SV = 1 | | | | |
| 408 | TRUE | FALSE | Uncharacterized protein | tr \| F6ZBT2 \| | 99 kDa | | 2 |
| | | | OS = Equus caballus | F6ZBT2_HORSE | | | |
| | | | GN = ABR PE = 4 SV = 1 | | | | |
| 409 | TRUE | FALSE | Uncharacterized protein | tr \| F6ZEQ3 \| | 51 kDa | TRUE | 2 |
| | | | OS = Equus caballus | F6ZEQ3_HORSE | | | |
| | | | GN = KRT16 PE = 3 SV = 1 | | | | |
| 410 | TRUE | FALSE | Uncharacterized protein | tr \| F6ZPC6 \| | 22 kDa | TRUE | 2 |
| | | | OS = Equus caballus | F6ZPC6_HORSE | | | |
| | | | GN = RAB1A PE = 3 SV = 1 | | | | |
| 411 | TRUE | FALSE | Uncharacterized protein | tr \| F7A0N8 \| | 65 kDa | | 2 |
| | | | OS = Equus caballus | F7A0N8_HORSE | | | |
| | | | GN = PPP2R1A PE = 4 SV = 1 | | | | |
| 412 | TRUE | FALSE | Uncharacterized protein | tr \| F7AH65 \| | 45 kDa | | 2 |
| | | | (Fragment) | F7AH65_HORSE | | | |
| | | | OS = Equus caballus | | | | |
| | | | GN = PSPC1 PE = 4 SV = 1 | | | | |
| 413 | TRUE | FALSE | Uncharacterized protein | tr \| F7B320 \| | 62 kDa | | 2 |
| | | | OS = Equus caballus | F7B320_HORSE | | | |
| | | | GN = DPYSL2 PE = 4 SV = 1 | | | | |
| 414 | TRUE | FALSE | Uncharacterized protein | tr \| F7B5P1 \| | 53 kDa | | 2 |
| | | | OS = Equus caballus | F7B5P1_HORSE | | | |
| | | | GN = CNDP2 PE = 4 SV = 1 | | | | |
| 415 | TRUE | FALSE | Guanine nucleotide-binding | tr \| F7BGR9 \| | 8 kDa | | 1 |
| | | | protein subunit gamma | F7BGR9_HORSE | | | |
| | | | OS = Equus caballus | | | | |
| | | | GN = GNG12 PE = 3 | | | | |
| 416 | TRUE | FALSE | Uncharacterized protein | tr \| F7BK52 \| | 37 kDa | | 2 |
| | | | OS = Equus caballus | F7BK52_HORSE | | | |
| | | | GN = CALU PE = 4 SV = 1 | | | | |
| 417 | TRUE | FALSE | Uncharacterized protein | tr \| F7BRC5 \| | 48 kDa | | 2 |
| | | | OS = Equus caballus | F7BRC5_HORSE | | | |
| | | | GN = PLVAP PE = 4 SV = 1 | | | | |
| 418 | TRUE | FALSE | Uncharacterized protein | tr \| F7C0E6 \| | 71 kDa | | 2 |
| | | | OS = Equus caballus | F7C0E6_HORSE | | | |
| | | | GN = PLS1 PE = 4 SV = 1 | | | | |
| 419 | TRUE | FALSE | Uncharacterized protein | tr \| F7C1X7 \| | 38 kDa | | 2 |
| | | | OS = Equus caballus | F7C1X7_HORSE | | | |
| | | | GN = SLC9A3R1 PE = 4 SV = 1 | (+1) | | | |
| 420 | TRUE | FALSE | Proteasome subunit beta type | tr \| F7CUX8 \| | 30 kDa | | 2 |
| | | | OS = Equus caballus | F7CUX8_HORSE | | | |
| | | | GN = PSMB7 PE = 3 SV = 1 | | | | |
| 421 | TRUE | FALSE | Uncharacterized protein | tr \| F7DV79 \| | 75 kDa | | 1 |
| | | | OS = Equus caballus | F7DV79_HORSE | | | |
| | | | GN = ESRP1 PE = 4 SV = 1 | | | | |
| 422 | TRUE | FALSE | Uncharacterized protein | tr \| F7DVX3 \| | 50 kDa | | 1 |
| | | | OS = Equus caballus | F7DVX3_HORSE | | | |
| | | | GN = RUVBL1 PE = 4 SV = 1 | | | | |
| 423 | TRUE | FALSE | Uncharacterized protein | tr \| F7DWC8 \| | 24 kDa | | 2 |
| | | | (Fragment) | F7DWC8_HORSE | | | |
| | | | OS = Equus caballus | | | | |
| | | | GN = ETFB PE = 4 SV = 1 | | | | |
| 424 | TRUE | FALSE | Uncharacterized protein | tr \| F7CPH9 \| | 30 kDa | | 2 |
| | | | (Fragment) | F7CPH9_HORSE | | | |
| | | | OS = Equus caballus | | | | |
| | | | GN = HADH PE = 4 SV = 1 | | | | |
| 425 | TRUE | FALSE | Uncharacterized protein | tr \| F6WJX1 \| | 79 kDa | | 2 |
| | | | OS = Equus caballus | F6WJX1_HORSE | | | |
| | | | GN = ZFP62 PE = 4 SV = 1 | | | | |
| 426 | TRUE | FALSE | Uncharacterized protein | tr \| F7BYU6 \| | 16 kDa | | 2 |
| | | | OS = Equus caballus | F7BYU6_HORSE | | | |
| | | | GN = RBP2 PE = 3 SV = 1 | | | | |
| 427 | TRUE | FALSE | Metalloproteinase inhibitor 3 | sp \| Q9TUL9 \| | 24 kDa | | 1 |
| | | | OS = Equus caballus | TIMP3_HORSE | | | |
| | | | GN = TIMP3 PE = 2 SV = 1 | | | | |
| 428 | TRUE | FALSE | Uncharacterized protein | tr \| F6X8M4 \| | 75 kDa | | 1 |
| | | | OS = Equus caballus | F6X8M4_HORSE | | | |
| | | | GN = ODF2L PE = 4 SV = 1 | | | | |
| 429 | TRUE | FALSE | Uncharacterized protein | REVERSE_tr \| | 30 kDa | | 1 |
| | | | OS = Equus caballus | F6QVI0 \| | | | |
| | | | GN = HSD17B13 PE = 3 | F6QVI0_HORSE-R | | | |
| 430 | TRUE | FALSE | Uncharacterized protein | REVERSE_tr \| | 74 kDa | | 1 |
| | | | (Fragment) | F6RDW9 \| | | | |
| | | | OS = Equus caballus | F6RDW9_HORSE-R | | | |
| | | | GN = RECQL PE = 4 SV = 1 | | | | |
| 431 | TRUE | FALSE | Uncharacterized protein | REVERSE_tr \| | 22 kDa | | 1 |
| | | | OS = Equus caballus | F7BI34 \| | | | |
| | | | GN = VSNL1 PE = 4 SV = 1 | F7BI34_HORSE-R | | | |
| 432 | TRUE | FALSE | Alcohol dehydrogenase class-3 | sp \| P19854 \| | 40 kDa | | 1 |
| | | | OS = Equus caballus | ADHX_HORSE | | | |
| | | | GN = ADH5 PE = 1 SV = 2 | | | | |
| 433 | TRUE | FALSE | Pyruvate carboxylase | tr \| F6PII6 \| | 130 kDa | | 1 |
| | | | OS = Equus caballus | F6PII6_HORSE | | | |
| | | | GN = PC PE = 3 SV = 1 | (+1) | | | |
| 434 | TRUE | FALSE | Uncharacterized protein | tr \| F6PSB8 \| | 27 kDa | | 1 |
| | | | (Fragment) | F6PSB8_HORSE | | | |
| | | | OS = Equus caballus | | | | |
| | | | GN = LASP1 PE = 4 SV = 1 | | | | |
| 435 | TRUE | FALSE | Uncharacterized protein | tr \| F6Q0M8 \| | 161 kDa | | 1 |
| | | | (Fragment) | F6Q0M8_HORSE | | | |
| | | | OS = Equus caballus | | | | |
| | | | GN = COL4A2 PE = 4 SV = 1 | | | | |
| 436 | TRUE | FALSE | Uncharacterized protein | tr \| F6Q8B3 \| | 23 kDa | | 1 |
| | | | OS = Equus caballus | F6Q8B3_HORSE | | | |
| | | | GN = RAB7A PE = 3 SV = 1 | | | | |
| 437 | TRUE | FALSE | Uncharacterized protein | tr \| F6QH40 \| | 60 kDa | | 1 |
| | | | (Fragment) | F6QH40_HORSE | | | |
| | | | OS = Equus caballus | (+3) | | | |
| | | | GN = SRC PE = 4 SV = 1 | | | | |
| 438 | TRUE | FALSE | Chaperone protein DnaJ | tr \| F6QSF7 \| | 45 kDa | | 1 |
| | | | OS = Equus caballus | F6QSF7_HORSE | | | |
| | | | GN = DNAJ PE = 3 SV = 1 | (+1) | | | |
| 439 | TRUE | FALSE | Uncharacterized protein | tr \| F6QYS3 \| | 61 kDa | | 1 |
| | | | OS = Equus caballus | F6QYS3_HORSE | | | |
| | | | GN = ECM1 PE = 4 SV = 1 | | | | |
| 440 | TRUE | FALSE | Uncharacterized protein | tr \| F6R0A9 \| | 22 kDa | | 1 |
| | | | OS = Equus caballus | F6R0A9_HORSE | | | |
| | | | PE = 4 SV = 1 | (+1) | | | |
| 441 | TRUE | FALSE | Uncharacterized protein | tr \| F6R5B2 \| | 24 kDa | TRUE | 1 |
| | | | OS = Equus caballus | F6R5B2_HORSE | | | |
| | | | GN = HMGB2 PE = 4 SV = 1 | | | | |
| 442 | TRUE | FALSE | Uncharacterized protein | tr \| F6R869 \| | 43 kDa | | 1 |
| | | | (Fragment) | F6R869_HORSE | | | |
| | | | OS = Equus caballus | | | | |
| | | | GN = STXBP3 PE = 4 SV = 1 | | | | |
| 443 | TRUE | FALSE | Uncharacterized protein | tr \| F6RGL2 \| | 350 kDa | | 1 |
| | | | (Fragment) | F6RGL2_HORSE | | | |
| | | | OS = Equus caballus | (+1) | | | |
| | | | GN = RANBP2 PE = 4 SV = 1 | | | | |
| 444 | TRUE | FALSE | Uncharacterized protein | tr \| F6RL28 \| | 40 kDa | | 1 |
| | | | OS = Equus caballus | F6RL28_HORSE | | | |
| | | | GN = RP2 PE = 4 SV = 1 | | | | |
| 445 | TRUE | FALSE | Uncharacterized protein | tr \| F6S0U0 \| | 26 kDa | | 1 |
| | | | OS = Equus caballus | F6S0U0_HORSE | | | |
| | | | GN = U2AF1L4 PE = 4 SV = 1 | (+1) | | | |
| 446 | TRUE | FALSE | Uncharacterized protein | tr \| F6SN62 \| | 72 kDa | | 1 |
| | | | (Fragment) | F6SN62_HORSE | | | |
| | | | OS = Equus caballus | | | | |
| | | | GN = DDX3X PE = 3 SV = 1 | | | | |
| 447 | TRUE | FALSE | Uncharacterized protein | tr \| F6T580 \| | 41 kDa | | 1 |
| | | | (Fragment) | F6T580_HORSE | | | |
| | | | OS = Equus caballus | | | | |
| | | | GN = PIP4K2A PE = 4 SV = 1 | | | | |
| 448 | TRUE | FALSE | Uncharacterized protein | tr \| F6T845 \| | 233 kDa | | 1 |
| | | | OS = Equus caballus | F6T845_HORSE | | | |
| | | | GN = MYO18A PE = 4 SV = 1 | | | | |
| 449 | TRUE | FALSE | Uncharacterized protein | tr \| F6TGA5 \| | 22 kDa | TRUE | 1 |
| | | | OS = Equus caballus | F6TGA5_HORSE | | | |
| | | | GN = RAB1B PE = 3 SV = 1 | | | | |
| 450 | TRUE | FALSE | Uncharacterized protein | tr \| F6TIM4 \| | 33 kDa | | 1 |
| | | | (Fragment) | F6TIM4_HORSE | | | |
| | | | OS = Equus caballus | | | | |
| | | | GN = FBL PE = 3 SV = 1 | | | | |
| 451 | TRUE | FALSE | Uncharacterized protein | tr \| F6TY61 \| | 30 kDa | | 1 |
| | | | (Fragment) | F6TY61_HORSE | | | |
| | | | OS = Equus caballus | | | | |
| | | | GN = DDAH2 PE = 4 SV = 1 | | | | |
| 452 | TRUE | FALSE | Serine/threonine-protein | tr \| F6U210 \| | 32 kDa | | 1 |
| | | | phosphatase (Fragment) | F6U210_HORSE | | | |
| | | | OS = Equus caballus | (+1) | | | |
| | | | GN = PPP2CA PE = 3 SV = 1 | | | | |
| 453 | TRUE | FALSE | Uncharacterized protein | tr \| F6UNR3 \| | 71 kDa | | 1 |
| | | | OS = Equus caballus | F6UNR3_HORSE | | | |
| | | | GN = EEF1D PE = 3 SV = 1 | | | | |
| 454 | TRUE | FALSE | Uncharacterized protein | tr \| F6V2B6 \| | 18 kDa | | 1 |
| | | | (Fragment) | F6V2B6_HORSE | | | |
| | | | OS = Equus caballus | | | | |
| | | | GN = CHMP4B PE = 4 SV = 1 | | | | |
| 455 | TRUE | FALSE | Uncharacterized protein | tr \| F6V2X7 \| | 84 kDa | | 1 |
| | | | OS = Equus caballus | F6V2X7_HORSE | | | |
| | | | GN = PLOD1 PE = 4 SV = 1 | | | | |
| 456 | TRUE | FALSE | Uncharacterized protein | tr \| F6V3K2 \| | 19 kDa | | 1 |
| | | | OS = Equus caballus | F6V3K2_HORSE | | | |
| | | | GN = SSR4 PE = 4 SV = 1 | | | | |
| 457 | TRUE | FALSE | Uncharacterized protein | tr \| F6VB94 \| | 73 kDa | | 1 |
| | | | (Fragment) | F6VB94_HORSE | | | |
| | | | OS = Equus caballus | | | | |
| | | | GN = TGFBI PE = 4 SV = 1 | | | | |
| 458 | TRUE | FALSE | Proteasome subunit alpha type | tr \| F6VEA3 \| | 26 kDa | | 1 |
| | | | OS = Equus caballus | F6VEA3_HORSE | | | |
| | | | GN = PSMA5 PE = 3 | | | | |
| 459 | TRUE | FALSE | Uncharacterized protein | tr \| F6VN96 \| | 76 kDa | | 1 |
| | | | OS = Equus caballus | F6VN96_HORSE | | | |
| | | | GN = CDH13 PE = 4 SV = 1 | | | | |
| 460 | TRUE | FALSE | Uncharacterized protein | tr \| F6VR95 \| | 79 kDa | | 1 |
| | | | OS = Equus caballus | F6VR95_HORSE | | | |
| | | | GN = NDUFS1 PE = 4 SV = 1 | | | | |
| 461 | TRUE | FALSE | Uncharacterized protein | tr \| F6VSL0 \| | 20 kDa | | 1 |
| | | | (Fragment) | F6VSL0_HORSE | | | |
| | | | OS = Equus caballus | | | | |
| | | | GN = RAN PE = 4 SV = 1 | | | | |
| 462 | TRUE | FALSE | Uncharacterized protein | tr \| F6VWA8 \| | 270 kDa | | 1 |
| | | | (Fragment) | F6VWA8_HORSE | | | |
| | | | OS = Equus caballus | | | | |
| | | | GN = IGF2R PE = 4 SV = 1 | | | | |
| 463 | TRUE | FALSE | Uncharacterized protein | tr \| F6W0T6 \| | 39 kDa | | 1 |
| | | | (Fragment) | F6W0T6_HORSE | | | |
| | | | OS = Equus caballus | | | | |
| | | | GN = SEPT9 PE = 3 SV = 1 | | | | |
| 464 | TRUE | FALSE | Uncharacterized protein | tr \| F6WBG0 \| | 24 kDa | | 1 |
| | | | OS = Equus caballus | F6WBG0_HORSE | | | |
| | | | GN = RPL13A PE = 3 SV = 1 | | | | |
| 465 | TRUE | FALSE | Uncharacterized protein | tr \| F6WQ69 \| | 41 kDa | | 1 |
| | | | OS = Equus caballus | F6WQ69_HORSE | | | |
| | | | GN = SEC62 PE = 4 SV = 1 | | | | |
| 466 | TRUE | FALSE | Uncharacterized protein | tr \| F6WXF6 \| | 67 kDa | | 1 |
| | | | OS = Equus caballus | F6WXF6_HORSE | | | |
| | | | GN = AIFM1 PE = 4 SV = 1 | | | | |
| 467 | TRUE | FALSE | Uncharacterized protein | tr \| F6WZ50 \| | 146 kDa | | 1 |
| | | | OS = Equus caballus | F6WZ50_HORSE | | | |
| | | | GN = SF3B1 PE = 4 SV = 1 | | | | |
| 468 | TRUE | FALSE | Uncharacterized protein | tr \| F6X7Q3 \| | 73 kDa | | 1 |
| | | | (Fragment) | F6X7Q3_HORSE | | | |
| | | | OS = Equus caballus | (+1) | | | |
| | | | GN = SDHA PE = 4 SV = 1 | | | | |
| 469 | TRUE | FALSE | Uncharacterized protein | tr \| F6XJF9 \| | 260 kDa | | 1 |
| | | | OS = Equus caballus | F6XJF9_HORSE | | | |
| | | | GN = TPR PE = 4 SV = 1 | (+1) | | | |
| 470 | TRUE | FALSE | Uncharacterized protein | tr \| F6XJR7 \| | 21 kDa | | 1 |
| | | | OS = Equus caballus | F6XJR7_HORSE | | | |
| | | | GN = CSRP1 PE = 4 SV = 1 | | | | |
| 471 | TRUE | FALSE | Uncharacterized protein | tr \| F6Y959 \| | 40 kDa | | 1 |
| | | | (Fragment) | F6Y959_HORSE | | | |
| | | | OS = Equus caballus | (+1) | | | |
| | | | GN = GNA11 PE = 4 SV = 1 | | | | |
| 472 | TRUE | FALSE | Uncharacterized protein | tr \| F6YNM7 \| | 79 kDa | | 1 |
| | | | (Fragment) | F6YNM7_HORSE | | | |
| | | | OS = Equus caballus | | | | |
| | | | GN = PREP PE = 4 SV = 1 | | | | |
| 473 | TRUE | FALSE | Uncharacterized protein | tr \| F6YRE7 \| | 69 kDa | | 1 |
| | | | OS = Equus caballus | F6YRE7_HORSE | | | |
| | | | GN = LTA4H PE = 4 SV = 1 | | | | |
| 474 | TRUE | FALSE | Uncharacterized protein | tr \| F6YWS5 \| | 49 kDa | | 1 |
| | | | OS = Equus caballus | F6YWS5_HORSE | | | |
| | | | GN = PSMC2 PE = 3 SV = 1 | | | | |
| 475 | TRUE | FALSE | T-complex protein 1 subunit | tr \| F6Z6Q7 \| | 54 kDa | | 1 |
| | | | delta (Fragment) | F6Z6Q7_HORSE | | | |
| | | | OS = Equus caballus | | | | |
| | | | GN = CCT4 PE = 3 SV = 1 | | | | |
| 476 | TRUE | FALSE | Uncharacterized protein | tr \| F6ZAG4 \| | 33 kDa | | 1 |
| | | | OS = Equus caballus | F6ZAG4_HORSE | | | |
| | | | GN = CPPED1 PE = 4 SV = 1 | | | | |
| 477 | TRUE | FALSE | Uncharacterized protein | tr \| F6ZJE4 \| | 24 kDa | TRUE | 1 |
| | | | OS = Equus caballus | F6ZJE4_HORSE | | | |
| | | | GN = RAB8A PE = 3 SV = 1 | | | | |
| 478 | TRUE | FALSE | Uncharacterized protein | tr \| F7AD76 \| | 13 kDa | | 1 |
| | | | OS = Equus caballus | F7AD76_HORSE | | | |
| | | | GN = RPL34 PE = 4 SV = 1 | (+1) | | | |
| 479 | TRUE | FALSE | Annexin | tr \| F7ALC9 \| | 50 kDa | | 1 |
| | | | OS = Equus caballus | F7ALC9_HORSE | | | |
| | | | GN = ANXA7 PE = 3 SV = 1 | | | | |
| 480 | TRUE | FALSE | Uncharacterized protein | tr \| F7AQH1 \| | 84 kDa | | 1 |
| | | | OS = Equus caballus | F7AQH1_HORSE | | | |
| | | | GN = EPB41L2 PE = 4 SV = 1 | (+2) | | | |
| 481 | TRUE | FALSE | Uncharacterized protein | tr \| F7ATL5 \| | 41 kDa | TRUE | 1 |
| | | | OS = Equus caballus | F7ATL5_HORSE | | | |
| | | | GN = KRT14 PE = 3 SV = 1 | | | | |
| 482 | TRUE | FALSE | Uncharacterized protein | tr \| F7AW05 \| | 22 kDa | | 1 |
| | | | OS = Equus caballus | F7AW05_HORSE | | | |
| | | | GN = CLEC3B PE = 4 SV = 1 | | | | |
| 483 | TRUE | FALSE | Uncharacterized protein | tr \| F7AXI9 \| | 25 kDa | | 1 |
| | | | (Fragment) | F7AXI9_HORSE | | | |
| | | | OS = Equus caballus | | | | |
| | | | GN = PRDX6 PE = 4 SV = 1 | | | | |
| 484 | TRUE | FALSE | Transaldolase (Fragment) | tr \| F7BA40 \| | 34 kDa | | 1 |
| | | | OS = Equus caballus | F7BA40_HORSE | | | |
| | | | GN = TALDO1 PE = 3 SV = 1 | | | | |
| 485 | TRUE | FALSE | Uncharacterized protein | tr \| F7BA85 \| | 102 kDa | | 1 |
| | | | OS = Equus caballus | F7BA85_HORSE | | | |
| | | | GN = HK1 PE = 3 SV = 1 | | | | |
| 486 | TRUE | FALSE | Uncharacterized protein | tr \| F7BB45 \| | 142 kDa | | 1 |
| | | | OS = Equus caballus | F7BB45_HORSE | | | |
| | | | GN = HDLBP PE = 4 SV = 1 | | | | |
| 487 | TRUE | FALSE | Uncharacterized protein | tr \| F7BJ30 \| | 91 kDa | | 1 |
| | | | OS = Equus caballus | F7BJ30_HORSE | | | |
| | | | GN = AP1G1 PE = 4 SV = 1 | | | | |
| 488 | TRUE | FALSE | Uncharacterized protein | tr \| F7BW76 \| | 18 kDa | | 1 |
| | | | OS = Equus caballus | F7BW76_HORSE | | | |
| | | | GN = GMFB PE = 4 SV = 1 | | | | |
| 489 | TRUE | FALSE | Uncharacterized protein | tr \| F7BZE6 \| | 51 kDa | | 1 |
| | | | OS = Equus caballus | F7BZE6_HORSE | | | |
| | | | GN = ILK PE = 4 SV = 1 | | | | |
| 490 | TRUE | FALSE | Uncharacterized protein | tr \| F7C7W3 \| | 35 kDa | | 1 |
| | | | OS = Equus caballus | F7C7W3_HORSE | | | |
| | | | GN = GNAI1 PE = 4 SV = 1 | | | | |
| 491 | TRUE | FALSE | Ubiquitin carboxyl-terminal | tr \| F7C9G5 \| | 96 kDa | | 1 |
| | | | hydrolase (Fragment) | F7C9G5_HORSE | | | |
| | | | OS = Equus caballus | | | | |
| | | | GN = USP5 PE = 3 SV = 1 | | | | |
| 492 | TRUE | FALSE | Uncharacterized protein | tr \| F7CC29 \| | 10 kDa | | 1 |
| | | | OS = Equus caballus | F7CC29_HORSE | | | |
| | | | GN = SNRPF PE = 4 SV = 1 | | | | |
| 493 | TRUE | FALSE | Uncharacterized protein | tr \| F7CGV8 \| | 105 kDa | | 1 |
| | | | (Fragment) | F7CGV8_HORSE | | | |
| | | | OS = Equus caballus | | | | |
| | | | GN = COL6A2 PE = 4 SV = 1 | | | | |
| 494 | TRUE | FALSE | Uncharacterized protein | tr \| F7CHJ5 \| | 86 kDa | | 1 |
| | | | (Fragment) | F7CHJ5_HORSE | | | |
| | | | OS = Equus caballus | | | | |
| | | | GN = PDXDC1 PE = 3 SV = 1 | | | | |
| 495 | TRUE | FALSE | Uncharacterized protein | tr \| F7CK08 \| | 14 kDa | | 1 |
| | | | OS = Equus caballus | F7CK08_HORSE | | | |
| | | | GN = SNRPD2 PE = 4 SV = 1 | | | | |
| 496 | TRUE | FALSE | Uncharacterized protein | tr \| F7D144 \| | 39 kDa | | 1 |
| | | | OS = Equus caballus | F7D144_HORSE | | | |
| | | | GN = CAPG PE = 4 SV = 1 | | | | |
| 497 | TRUE | FALSE | Uncharacterized protein | tr \| F7D3H6 \| | 26 kDa | | 1 |
| | | | OS = Equus caballus | F7D3H6_HORSE | | | |
| | | | GN = RALB PE = 4 SV = 1 | | | | |
| 498 | TRUE | FALSE | Uncharacterized protein | tr \| F7DCX4 \| | 61 kDa | | 1 |
| | | | OS = Equus caballus | F7DCX4_HORSE | | | |
| | | | GN = SLC25A20 PE = 3 SV = 1 | | | | |
| 499 | TRUE | FALSE | Uncharacterized protein | tr \| F7DLB5 \| | 39 kDa | TRUE | 1 |
| | | | OS = Equus caballus | F7DLB5_HORSE | | | |
| | | | GN = ERLIN1 PE = 4 SV = 1 | | | | |
| 500 | TRUE | FALSE | Uncharacterized protein | tr \| F7DZH1 \| | 100 kDa | | 1 |
| | | | (Fragment) | F7DZH1_HORSE | | | |
| | | | OS = Equus caballus | | | | |
| | | | GN = SYNPO2L PE = 4 SV = 1 | | | | |
| 501 | TRUE | FALSE | Uncharacterized protein | tr \| F7E306 \| | 49 kDa | | 1 |
| | | | OS = Equus caballus | F7E306_HORSE | | | |
| | | | GN = SEPT11 PE = 3 SV = 1 | | | | |
| 502 | TRUE | FALSE | Uncharacterized protein | REVERSE_tr \| | 50 kDa | | 1 |
| | | | OS = Equus caballus | F6QWY6 \| | | | |
| | | | GN = SH3BP5 PE = 4 SV = 1 | F6QWY6_HORSE-R | | | |
| 503 | TRUE | FALSE | sp \| CATA_HUMAN \| | sp \| CATA_HUMAN \| | 60 kDa | | 1 |
| | | | | (+1) | | | |
| 504 | TRUE | FALSE | Uncharacterized protein | tr \| F6ZJP9 \| | 50 kDa | | 1 |
| | | | OS = Equus caballus | F6ZJP9_HORSE | | | |
| | | | GN = LCAT PE = 4 SV = 1 | | | | |
| 505 | TRUE | FALSE | Uncharacterized protein | tr \| F6WVD1 \| | 8 kDa | | 1 |
| | | | OS = Equus caballus | F6WVD1_HORSE | | | |
| | | | GN = RPS28 PE = 4 SV = 1 | | | | |
| 506 | TRUE | FALSE | Uncharacterized protein | tr \| F6YE64 \| | 25 kDa | | 1 |
| | | | OS = Equus caballus | F6YE64_HORSE | | | |
| | | | GN = SRSF9 PE = 4 SV = 1 | | | | |
| 507 | TRUE | FALSE | Uncharacterized protein | tr \| F7DZZ9 \| | 40 kDa | | 1 |
| | | | (Fragment) | F7DZZ9_HORSE | | | |
| | | | OS = Equus caballus | | | | |
| | | | GN = VASP PE = 4 SV = 1 | | | | |
| 508 | TRUE | FALSE | Uncharacterized protein | tr \| F7A6J6 \| | 14 kDa | | 1 |
| | | | OS = Equus caballus | F7A6J6_HORSE | | | |
| | | | GN = HRSP12 PE = 4 SV = 1 | | | | |
| 509 | TRUE | FALSE | Uncharacterized protein | tr \| F6PLW0 \| | 84 kDa | | 1 |
| | | | (Fragment) | F6PLW0_HORSE | | | |
| | | | OS = Equus caballus | (+2) | | | |
| | | | GN = DNM3 PE = 3 SV = 1 | | | | |
| 510 | TRUE | FALSE | Uncharacterized protein | tr \| F6XSG4 \| | 127 kDa | | 1 |
| | | | (Fragment) | F6XSG4_HORSE | | | |
| | | | OS = Equus caballus | (+1) | | | |
| | | | GN = ATP2B1 PE = 3 SV = 1 | | | | |
| 511 | TRUE | FALSE | 40S ribosomal protein S24 | tr \| F6Z3G3 \| | 15 kDa | | 1 |
| | | | (Fragment) | F6Z3G3_HORSE | | | |
| | | | OS = Equus caballus | | | | |
| | | | GN = RPS24 PE = 3 SV = 1 | | | | |
| 512 | TRUE | FALSE | Uncharacterized protein | REVERSE_tr \| | 91 kDa | | 1 |
| | | | (Fragment) | F6VG41 \| | | | |
| | | | OS = Equus caballus | F6VG41_HORSE-R | | | |
| | | | GN = HIF1A PE = 4 SV = 1 | | | | |
| 513 | TRUE | FALSE | Uncharacterized protein | tr \| F6V2P8 \| | 23 kDa | | 1 |
| | | | OS = Equus caballus | F6V2P8_HORSE | | | |
| | | | GN = RAB25 PE = 3 SV = 1 | | | | |
| 514 | TRUE | FALSE | Uncharacterized protein | tr \| F6PWL9 \| | 50 kDa | | 1 |
| | | | (Fragment) | F6PWL9_HORSE | | | |
| | | | OS = Equus caballus | | | | |
| | | | GN = GDI1 PE = 4 SV = 1 | | | | |
| 515 | TRUE | FALSE | Uncharacterized protein | tr \| F6RKM7 \| | 15 kDa | | 1 |
| | | | OS = Equus caballus | F6RKM7_HORSE | | | |
| | | | GN = RPL22 PE = 4 SV = 1 | | | | |
| 516 | TRUE | FALSE | Proteasome subunit alpha type | tr \| F6TCI4 \| | 26 kDa | | 1 |
| | | | OS = Equus caballus | F6TCI4_HORSE | | | |
| | | | GN = LOC100064293 PE = 3 | | | | |

What is claimed is:

1. A method of harvesting, processing and storing a plurality of proteins from a mammalian feto-placental unit comprising:
dissecting a mammalian uterus to harvest at least one component of the mammalian feto-placental unit;
blast freezing the component; and
storing the blast frozen component;
wherein the blast frozen component includes the proteins.

2. The method of claim 1, further comprising:
lyophilizing the blast frozen component to remove at least some water from the blast frozen component thereby creating a freeze-dried form; and
storing the lyophilized component;
wherein the lyophilized component includes the proteins.

3. The method of claim 1, wherein the proteins comprise at least one essential fragment of at least one of the proteins.

4. The method of claim 1, wherein the proteins comprise a plurality of chaperone proteins.

5. The method of claim 1, wherein the proteins are selected from the group consisting of Serum albumin, Actin, cytoplasmic 1, I alpha globin, Hemoglobin fetal subunit beta, Vimentin, Beta globin chain, TPM1, Annexin A2, Protein disulfide isomerase family A member 3, Alpha-2-HS-glycoprotein, Fatty acid binding protein 5, Cofilin-1, 78 kDa glucose-regulated protein, Gelsolin isoform b, Beta-A globin chain, Glyceraldehyde-3-phosphate dehydrogenase, Heat shock protein alpha, Heat shock protein 70, Peptidylprolyl isomerase A, 14-3-3 protein zeta/delta, Histone H3, Peroxiredoxin 2, Cathepsin D, Uterine milk protein, Tubulin beta chain, Myosin light chain 6, Endoplasmic reticulum protein 29, Tubulin alpha chain, Solute carrier family 2, facilitated glucose transporter member 1, Alpha-1-antitrypsin transcript variant 1, Heat shock protein 10, Pregnancy-associated glycoprotein 3, Hemoglobin subunit beta, Isocitrate dehydrogenase [NADP] cytoplasmic, Elongation factor-1 alpha, Phosphoglycerate kinase, 14-3-3 protein epsilon, Putative tropomyosin, Tumor protein translationally-controlled 1, Galectin-1, Transaldolase 1, Pregnancy-associated glycoprotein 4, Pregnancy-associated glycoprotein 1, Sodium/potassium-transporting ATPase subunit alpha-1, Lamin B1, pregnancy-associated glycoprotein 6, 14-3-3 protein beta/alpha, metallopeptidase inhibitor 2, fatty acid binding protein 5, Myosin regulatory light chain MRCL3, Transferrin, Enolase 1, Cathelicidin-1, 6-phosphogluconate dehydrogenase decarboxylating, Elongation factor-1 alpha, ATP-citrate synthase, Ribosomal protein S8, Pyruvate kinase, Pre-mRNA splicing factor SRP20-like protein, alpha 2, 5 prime, Malate dehydrogenase, Cystatin-B, Chorionic somatomammotropin hormone, Carbonic anhydrase 2, SLC25A6, Decorin, 60S ribosomal protein L6, Protein disulfide isomerase-associated 4, Pregnancy-associated glycoprotein 11, Prostaglandin F synthase, Integrin beta-1, H+ transporting ATP synthase subunit D, RHOA, Adenylate kinase, Lactate dehydrogenase A, RAB10, Glucose-6-phosphate 1-dehydrogenase, Elongation factor 1-delta, Ribosomal protein S17, Insulin-like growth factor-binding protein-7, Ribosomal protein L19, ATP synthase alpha subunit, RAC1, Calpain II 80 kDa subunit, Secreted phosphoprotein 24, CD9 antigen, Aspartate aminotransferase, DNA mismatch repair protein MutL, Ribosomal protein S6, 14-3-3 protein gamma LDHA protein, Putative peptidase, Myosin light chain kinase, smooth muscle, cAMP-dependent protein kinase regulatory subunit alpha 1, Elongation factor 1-alpha, Actin, GNAZ, Eukaryotic translation initiation factor 5A, Mitochondrial bifunctional methylenetetrahydrofolate dehydrogenase/cyclohydrolase, Solute carrier family 2, facilitated glucose transporter member 3, Thioredoxin, ADP-ribosylation factor 1, NADH dehydrogenase (Ubiquinone) 1 beta, Proteasome subunit alpha type, 2, Gamma fibrinogen, Putative H-ATPase subunit B, Proteasome subunit alpha type, 60S ribosomal protein L10, 14-3-3 protein sigma, Chaperone protein DnaK, Ribosomal protein s15, Putative uncharacterized protein, Aspartyl-tRNA synthetase, and Proteasome subunit alpha.

6. The method of claim 1, further comprising using at least a portion of the proteins for medicinal purposes.

7. The method of claim 6, wherein the using step comprises treating for a disease or aging in a mammalian subject.

8. The method of claim 7, wherein the treating step comprises administering the portion of the proteins to the mammalian subject with a sublingual procedure.

9. The method of claim 7, wherein the treating step comprises administering the portion of the proteins to the mammalian subject with an intra-ocular procedure

10. The method of claim 7, wherein the treating step comprises administering the portion of the proteins to the mammalian subject with an intra-rectal procedure.

11. The method of claim 7, wherein the treating step comprises:
reconstituting the portion of the proteins of the lyophilized component with a fluid; and
administering the reconstituted portion of the proteins to the mammalian subject.

12. The method of claim 11, wherein the administering step comprises administering the reconstituted portion of the proteins to the mammalian subject through a procedure selected from the group consisting of an oral administration, a rectal administration, a cutaneous administration, a subcutaneous administration, an intravenous injection, and an intramuscular injection.

13. The method of claim 1 wherein the component is selected from the group consisting of a liver component, a spleen component, a whole brain component, an ocular component, a gastro-intestinal component, a female specific component, and a male specific component.

14. A composition comprising:
a plurality of proteins from at least one lyophilized, blast frozen component of a harvested mammalian feto-placental unit reconstituted with a fluid.

15. The composition of claim 14, wherein the proteins comprise at least one essential fragment of at least one of the proteins.

16. The composition of claim 14, wherein the proteins comprise a plurality of chaperone proteins.

17. The composition of claim 14 wherein the lyophilized, blast frozen component is selected from the group consisting of a placenta-cord fetal component, a liver component, a spleen component, a whole brain component, an ocular component, a gastro-intestinal component, a female specific component, and a male specific component.

18. A method of treatment of a disease or aging in a mammalian subject comprising:
administering to the mammalian subject a plurality of proteins from at least one blast frozen component harvested from a mammalian feto-placental unit; and
reducing an accumulation of at least one intracellular protein in the mammalian subject.

19. The method of claim 18, wherein the proteins comprise at least one essential fragment of at least one of the proteins.

20. The method of claim 18 wherein the proteins comprise a plurality of chaperone proteins.

21. The method of claim 18, wherein the step of reducing the accumulation of the intracellular protein comprises at least one of folding, refolding, degradation and transfer across a cellular membrane of the intracellular protein.
